SEQUENCE LISTING (9 sequences)

SEQ ID NO: 1 - protein, 342 aa, Pseudozyma antarctica
MKLLSLTGVAGVLATCVAATPLVKRLPSGSDPAFSQPKSVLDAGLTCQGASPSSVSKPILLVPGTGTTGPQSFDSNWIPLSTQLGYTPCWISPPPFMLNDTQVNTEYMVNAITALYAGSGNNKLPVLTWSQGGLVAQWGLTFFPSIRSKVDRLMAFAPDYKGTVLAGPLDALAVSAPSVWQQTTGSALTTALRNAGGLTQIVPTTNLYSATDEIVQPQVSNSPLDSSYLFNGKNVQAQAVCGPLFVIDHAGSLTSQFSYVVGRSALRSTTGQARSADYGITDCNPLPANDLTPEQKVAAAALLAPAAAAIVAGPKQNCEPDLMPYARPFAVGKRTCSGIVTP

SEQ ID NO: 2 - protein, 549 aa, Diutina rugosa
MELALALSLIASVAAAPTATLANGDTITGLNAIINEAFLGIPFAEPPVGNLRFKDPVPYSGSLDGQKFTSYGPSCMQQNPEGTYEENLPKAALDLVMQSKVFEAVSPSSEDCLTINVVRPPGTKAGANLPVMLWIFGGGFEVGGTSTFPPAQMITKSIAMGKPIIHVSVNYRVSSWGFLAGDEIKAEGSANAGLKDQRLGMQWVADNIAAFGGDPTKVTIFGESAGSMSVMCHILWNDGDNTYKGKPLFRAGIMQSGAMVPSDAVDGIYGNEIFDLLASNAGCGSASDKLACLRGVSSDTLEDATNNTPGFLAYSSLRLSYLPRPDGVNITDDMYALVREGKYANIPVIIGDQNDEGTFFGTSSLNVTTDAQAREYFKQSFVHASDAEIDTLMTAYPGDITQGSPFDTGILNALTPQFKRISAVLGDLGFTLARRYFLNHYTGGTKYSFLSKQLSGLPVLGTFHSNDIVFQDYLLGSGSLIYNNAFIAFATDLDPNTAGLLVKWPEYTSSSQSGNNLMMINALGLYTGKDNFRTAGYDALFSNPPSFFV

SEQ ID NO: 3 - protein, 548 aa, Diutina rugosa
MKLCLLALGAAVAAAPTATLANGDTITGLNAIVNEKFLGIPFAEPPVGTLRFKPPVPYSASLNGQQFTSYGPSCMQMNPMGSFEDTLPKNARHLVLQSKIFQVVLPNDEDCLTINVIRPPGTRASAGLPVMLWIFGGGFELGGSSLFPGDQMVAKSVLMGKPVIHVSMNYRVASWGFLAGPDIQNEGSGNAGLHDQRLAMQWVADNIAGFGGDPSKVTIYGESAGSMSTFVHLVWNDGDNTYNGKPLFRAAIMQSGCMVPSDPVDGTYGTEIYNQVVASAGCGSASDKLACLRGLSQDTLYQATSDTPGVLAYPSLRLSYLPRPDGTFITDDMYALVRDGKYAHVPVIIGDQNDEGTLFGLSSLNVTTDAQARAYFKQSFIHASDAEIDTLMAAYTSDITQGSPFDTGIFNAITPQFKRISALLGDLAFTLARRYFLNYYQGGTKYSFLSKQLSGLPVLGTFHGNDIIWQDYLVGSGSVIYNNAFIAFANDLDPNKAGLWTNWPTYTSSSQSGNNLMQINGLGLYTGKDNFRPDAYSALFSNPPSFFV

SEQ ID NO: 4 - protein, 549 aa, Diutina rugosa
MKLALALSLIASVAAAPTAKLANGDTITGLNAIINEAFLGIPFAEPPVGNLRFKDPVPYSGSLNGQKFTSYGPSCMQQNPEGTFEENLGKTALDLVMQSKVFQAVLPQSEDCLTINVVRPPGTKAGANLPVMLWIFGGGFEIGSPTIFPPAQMVTKSVLMGKPIIHVAVNYRVASWGFLAGDDIKAEGSGNAGLKDQRLGMQWVADNIAGFGGDPSKVTIFGESAGSMSVLCHLIWNDGDNTYKGKPLFRAGIMQSGAMVPSDPVDGTYGNEIYDLFVSSAGCGSASDKLACLRSASSDTLLDATNNTPGFLAYSSLRLSYLPRPDGKNITDDMYKLVRDGKYASVPVIIGDQNDEGTIFGLSSLNVTTNAQARAYFKQSFIHASDAEIDTLMAAYPQDITQGSPFDTGIFNAITPQFKRISAVLGDLAFIHARRYFLNHFQGGTKYSFLSKQLSGLPIMGTFHANDIVWQDYLLGSGSVIYNNAFIAFATDLDPNTAGLLVNWPKYTSSSQSGNNLMMINALGLYTGKDNFRTAGYDALMTNPSSFFV

SEQ ID NO: 5 - protein, 549 aa, Diutina rugosa
MKLALVLSLIVSVAAAPTATLANGDTITGLNAIINEAFLGIPFAQPPVGNLRFKPPVPYSASLNGQKFTSYGPSCMQMNPLGNWDSSLPKAAINSLMQSKLFQAVLPNGEDCLTINVVRPSGTKPGANLPVMVWIFGGGFEVGGSSLFPPAQMITASVLMGKPIIHVSMNYRVASWGFLAGPDIKAEGSGNAGLHDQRLGLQWVADNIAGFGGDPSKVTIFGESAGSMSVMCQLLWNDGDNTYNGKPLFRAAIMQSGAMVPSDPVDGPYGTQIYDQVVASAGCGSASDKLACLRSISNDKLFQATSDTPGALAYPSLRLSFLPRPDGTFITDDMFKLVRDGKCANVPVIIGDQNDEGTVFALSSLNVTTDAQARQYFKESFIHASDAEIDTLMAAYPSDITQGSPFDTGIFNAITPQFKRIAAVLGDLAFTLPRRYFLNHFQGGTKYSFLSKQLSGLPVIGTHHANDIVWQDFLVSHSSAVYNNAFIAFANDLDPNKAGLLVNWPKYTSSSQSGNNLLQINALGLYTGKDNFRTAGYDALFTNPSSFFV

SEQ ID NO: 6 - protein, 549 aa, Diutina rugosa
MKLALALSLIASVAAAPTATLANGDTITGLNAIINEAFLGIPFAEPPVGNLRFKDPVPYRGSLNGQSFTAYGPSCMQQNPEGTYEENLPKVALDLVMQSKVFQAVLPNSEDCLTINVVRPPGTKAGANLPVMLWIFGGGFEIGSPTIFPPAQMVSKSVLMGKPIIHVAVNYRLASFGFLAGPDIKAEGSSNAGLKDQRLGMQWVADNIAGFGGDPSKVTIFGESAGSMSVLCHLLWNGGDNTYKGKPLFRAGIMQSGAMVPSDPVDGTYGTQIYDTLVASTGCSSASNKLACLRGLSTQALLDATNDTPGFLSYTSLRLSYLPRPDGANITDDMYKLVRDGKYASVPVIIGDQNDEGFLFGLSSLNTTTEADAEAYLRKSFIHATDADITALKAAYPSDVTQGSPFDTGILNALTPQLKRINAVLGDLTFTLSRRYFLNHYTGGPKYSFLSKQLSGLPILGTFHANDIVWQHFLLGSGSVIYNNAFIAFATDLDPNTAGLSVQWPKSTSSSQAGDNLMQISALGLYTGKDNFRTAGYNALFADPSHFFV

SEQ ID NO: 7 - protein, 392 aa, Rhizopus niveus
MVSFISISQGVSLCLLVSSMMLGSSAVPVSGKSGSSNTAVSASDNAALPPLISSRCAPPSNKGSKSDLQAEPYNMQKNTEWYESHGGNLTSIGKRDDNLVGGMTLDLPSDAPPISLSSSTNSASDGGKVVAATTAQIQEFTKYAGIAATAYCRSVVPGNKWDCVQCQKWVPDGKIITTFTSLLSDTNGYVLRSDKQKTIYLVFRGTNSFRSAITDIVFNFSDYKPVKGAKVHAGFLSSYEQVVNDYFPVVQEQLTAHPTYKVIVTGHSLGGAQALLAGMDLYQREPRLSPKNLSIFTVGGPRVGNPTFAYYVESTGIPFQRTVHKRDIVPHVPPQSFGFLHPGVESWIKSGTSNVQICTSEIETKDCSNSIVPFTSILDHLSYFDINEGSCL

SEQ ID NO: 8 - protein, 418 aa, Aspergillus niger (strain ATCC 1015)
MYIPSVLLLAASLFHGATALPTPGSTPIPPSQDPWYSAPEGFEEADPGAILRVRPAPGNLTVVVGNASAAYNILYRTTDSQYKPSWAVTTLLVPPVAASAAVNQSVLLSYQIAYDSFDVNASPSYAMYTSPPSDIILALQRGWFVNVPDYEGPNASFTAGVQSGHATLDSVRSVLASGFGLNEDAQYALWGYSGGALASEWAAELQMQYAPELNIAGLAVGGLTPNVTSVMDTVTSTISAGLIPAAALGLSSQHPETYEFILSQLKTTGPYNRTGFLAAKDLTLSEAEVFYAFQNIFDYFVNGSATFQAEVVQKALNQDGYMGYHGFPQMPVLAYKAIHDEISPIQDTDRVIKRYCGLGLNILYERNTIGGHSAEQVNGNARAWNWLTSIFDGTYAQQYKTEGCTIRNVTLNTTSSVY

SEQ ID NO: 9 - protein, 259 aa, Penicillium camemberti FM 013
MATIETQGNEDAFKPYDQFLLFGDSITQMACNQELGFAFHAGLQESYSRRLDVINRGLAGYSTAHAVKVFDKFFPSPQTANVRFMTIFFGANDACVPTHNQHVPLDQYKENLKTIIQHPATRAQNPRLILISPPPVNEHQLEAFDAAKDTPFPSRTASFTKSYAVAACEVGASLNIPVVDLWSAFMKPTGWKEGEPLIGARDVPSNDTLASLLTDGLHLTPAGNRIVYDELMKVIQANWPDQTPEVLPMVFPSWGDAPK

(19) United States
(12) Patent Application Publication
(10) Pub. No.: US 2021/0155653 A1
(43) Pub. Date: May 27, 2021

(54) METHOD FOR HOMOGENIZING BILE ACID DERIVATIVES

(71) Applicant: Enzymicals AG, Greifswald (DE)

(72) Inventors: Ulf Menyes, Neu Boltenhagen (DE); Philipp Suss, Greifswald (DE); Henrike Brundiek, Greifswald (DE); Melinda Fekete, Greifswald (DE)

(21) Appl. No.: 16/625,967

(22) PCT Filed: Jun. 21, 2018
(86) PCT No.: PCT/EP2018/066625

§ 371 Date: Dec. 23, 2019

(30) Foreign Application Priority Data
Jun. 22, 2017 (EP) 102017210542.9

(51) Int. Cl. C07J 9/00; C07J 31/00; C12P 33/00

(52) U.S. Cl. CPC C07J 9/005; C12P 33/00; C07J 31/003

(57) ABSTRACT

The present invention relates to a process for producing bile acid derivatives having a protected hydroxyl group in the 3 position comprising contacting a bile acid derivative having an unprotected 3-alpha-hydroxyl group with a specific lipase. The present invention further relates to a bile acid derivative obtained or obtainable by the process, to the use of the bile acid derivative obtained or obtainable by the process for producing lithocholic acid and also to a process for producing lithocholic acid and to lithocholic obtained by the process. The invention further relates to the use of lithocholic acid obtained or obtainable by the process for producing ursodeoxycholic acid or ursodeoxycholic acid derivatives.

The present invention relates to a process for producing bile acid derivatives having a protected hydroxyl group in the 3 position comprising contacting a bile acid derivative having an unprotected 3-alpha-hydroxyl group with a specific lipase.

A very wide variety of pharmaceutical products are nowadays produced from bile acids and the inputs are obtained from mammals. The employed animal starting material is composed of different bile acids on a species-specific basis, the bile acids differing from one another in terms of their hydroxylation pattern. The commonality is the beta configuration of the A-ring and the 3-alpha-hydroxy group on the A-ring. The bile acid mixtures obtained from pigs comprise for example chenodeoxycholic acid (CDCA), hyodeoxycholic acid (HDCA) and hyocholic acid (HCA). Virtually pure bile acids are available from only a few animal species. Thus for example cholic acid (CA) is obtainable from cattle without association with other bile acids. However, due to the high demand for pharmaceutical applications this is insufficient as the single source for pure bile acids. That use of bile acids from other animals has thus hitherto always required cleanly separating the various mixtures from one another, usually with considerable separation complexity.

The invention accordingly has for its object the provision of a process by which mixtures of different bile acids can be converted into one (uniform) base compound.

It was found that, surprisingly, such a process may be provided by selective enzymatic esterification of the 3-alpha-hydroxyl group on the A-ring of specific bile acid derivatives.

The present invention therefore relates to a process for producing bile acid derivatives having a protected hydroxyl group in the 3 position comprising:
i) providing a first composition comprising at least one bile acid derivative of general formula I:

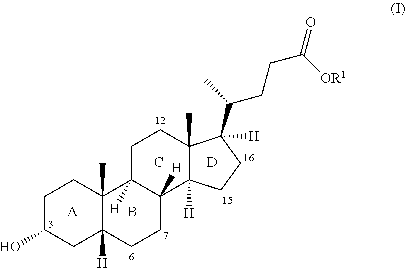

wherein the radical R¹ is selected from the group consisting of C1- to C30-alkyl group, C1- to C30-alkenyl group, C1- to C30-alkynyl group, C5- to C12-cycloalkyl group and C5- to C12-aryl group and at least one of the rings B and D has at least one further, preferably alpha-, hydroxyl group (positions 6, 7, 15, 16);
ii) contacting the first composition comprising at least one bile acid derivative of general formula I from i) with
   a compound R²—X, wherein R² is a —C(═O)—C1- to C30-alkyl group and X is selected from the group consisting of hydroxyl group, —O—C1- to C20-alkyl group, —O—C1- to C20-alkenyl group, —O—C1- to C20-alkynyl group; thiol group, —S—C1- to C20-alkyl group, amine group, —NHR³ group, —NR³R⁴ group, wherein R³ and R⁴ are each independently a C1- to C20-alkyl group, halogen atom and —O—(C═O)—R⁵ group, wherein R⁵ is a C1- to C30-alkyl group; and
   a lipase selected from the group consisting of lipase B from Candida antarctica of SEQ ID no. 1, lipase 1 from Diutina rugosa of SEQ ID no. 2, lipase 2 from Diutina rugosa of SEQ ID no. 3, lipase 3 from Diutina rugosa of SEQ ID no. 4, lipase 4 from Diutina rugosa of SEQ ID no. 5, lipase 5 from Diutina rugosa of SEQ ID no. 6, lipase from Rhizopus niveus of SEQ ID no. 7, lipase from Aspergillus niger (ATCC 1015) of SEQ ID no. 8 and lipase from Penicillium camemberti FM 013 of SEQ ID no. 9 or a homologous enzyme having a sequence identity of at least 65% with one of the sequences of SEQ ID no. 1 to SEQ ID no. 9 and having the same function as the lipase of SEQ ID no. 1 to SEQ ID no. 9,
   to obtain a second composition comprising at least one bile acid derivative of general formula II:

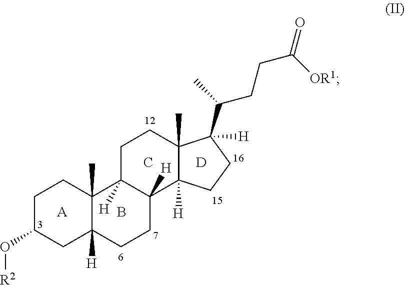

wherein the radical R¹ is as defined at i) for formula I and the radical R² is as defined at ii) and at least one of the rings B and D has at least one further, preferably alpha-, hydroxyl group.

The method of selective 3′OH esterification allows all bile acid derivatives, in particular all bile acid derivatives of general formula I), to be uniformized via chemical processes into a basic compound from which all desired bile acid species are obtainable by stereo- and enantioselective hydroxylation processes. This uniformization strategy makes it possible to meet the worldwide raw material demand of animal bile acids and reduce byproduct/waste production.

C5- to C12-cycloalkyl groups/C5- to C7-cycloalkyl groups comprise one ring system or two or more ring systems, wherein two or more ring systems are separated or annelated. C5- to C12-aryl groups comprise one ring system or two or more ring systems, wherein two or more ring systems are separated or annelated. Unless otherwise explicitly stated the term “alkyl” refers to branched and unbranched alkyl groups and the same applies to “alkenyl” and “Alkynyl”.

It is preferable when the radical R¹ of the bile acid derivative of general formula I/of the bile acid derivative of general formula II is selected from the group consisting of C1- to C18-alkyl group, C5- to C7-cycloalkyl group and phenyl group, more preferably of C1- to C5-alkyl group, and it is more preferable when R¹ is an unbranched C1- to C3-alkyl radical, more preferably a methyl radical.

The radical X of the compound R²—X in ii) is preferably selected from the group consisting of hydroxyl group, —O—C1- to C20-alkyl group, —O—C1- to C20-alkenyl group, —O—C1- to C20-alkynyl group, thiol group, —S—C1- to C20-alkyl group, amine group, —NHR³ group and —NR³R⁴ group, wherein R³ and R⁴ are each independently a C1- to C20-alkyl group; X is preferably selected from the group consisting of hydroxyl group, —O—C1- to C20-alkyl group and —O—C1- to C20-alkenyl group.

In the compound R²—X in ii)/in the bile acid derivative of general formula II R² is preferably an unbranched —C(═O)—C1- to C18-alkyl group, more preferably an unbranched —C(═O)—C1- to C5-alkyl group, more preferably —C(═O)—CH₃.

Particularly preferably employed as compound R²—X in ii) is a compound from the group consisting of C1- to C18-alkyl-C(═O)—O—C1- to C20-alkyl compound (alkyl carboxylates), C1- to C18-alkyl-C(═O)—O—C1- to C20-alkenyl compound, C1- to C18-alkyl-C(═O)—OH compound (carboxylic acid) and mixtures of two or more of these compounds. Most preferably employed as compound R²—X in ii) are ethyl acetate (acetic acid ethyl ester), vinyl acetate, acetic acid or mixtures of two or more of these compounds, more preferably ethyl acetate, vinyl acetate or a mixture of ethyl acetate and vinyl acetate.

Step ii) employs a lipase selected from the group consisting of lipase B from Candida antarctica of SEQ ID no. 1, lipase 1 from Diutina rugosa of SEQ ID no. 2, lipase 2 from Diutina rugosa of SEQ ID no. 3, lipase 3 from Diutina rugosa of SEQ ID no. 4, lipase 4 from Diutina rugosa of SEQ ID no. 5, lipase 5 from Diutina rugosa of SEQ ID no. 6, lipase from Rhizopus niveus of SEQ ID no. 7, lipase from Aspergillus niger (ATCC 1015) of SEQ ID no. 8 and lipase from Penicillium camemberti FM 013 of SEQ ID no. 9 or a homologous enzyme having a sequence identity of at least 65% with one of the sequences of SEQ ID no. 1 to SEQ ID no. 9 and having the same function as the lipase of SEQ ID no. 1 to SEQ ID no. 9. The lipases of SEQ ID nos. 1 to 9 are listed hereinbelow in table 1. All lipases are known, are available in public collections and are readily obtainable. The sequences of all lipases are listed in a very wide variety of databases and the contents of table 1 which follows correspond to the sequence protocol of those in the database UniProt (Status: 10 Jan. 2017).

TABLE 1
Lipases of SEQ ID nos. 1 to 9

| | | | SEQ |
|---|---|---|---|
| Name | Organism | Sequence | ID no. |
| Lipase B | Pseudozyma | >sp\|P41365\|LIPB_PSEA2 Lipase B | 1 |
| | antarctica | OS = Pseudozyma antarctica | |
| | (Moesziomyces | PE = 1 SV = 1 | |
| | antarcticus, | MKLLSLTGVAGVLATCVAATPLVKRLPSGSDPAFSQ | |
| | Candida | PKSVLDAGLTCQGASPSSVSKPILLVPGTGTTGPQS | |
| | antarctica) | FDSNWIPLSTQLGYTPCWISPPPFMLNDTQVNTEYM | |
| | | VNAITALYAGSGNNKLPVLTWSQGGLVAQWGLTFFP | |
| | | SIRSKVDRLMAFAPDYKGTVLAGPLDALAVSAPSVW | |
| | | QQTTGSALTTALRNAGGLTQIVPTTNLYSATDEIVQ | |
| | | PQVSNSPLDSSYLFNGKNVQAQAVCGPLFVIDHAGS | |
| | | LTSQFSYVVGRSALRSTTGQARSADYGITDCNPLPA | |
| | | NDLTPEQKVAAAALLAPAAAAIVAGPKQNCEPDLMP | |
| | | YARPFAVGKRTCSGIVTP | |
| Lipase 1 | Diutina rugosa | >sp\|P20261\|LIP1_DIURU Lipase 1 | 2 |
| | (Candida | OS = Diutina rugosa | |
| | rugosa) | GN = LIP1 PE = 1 SV = 3 | |
| | | MELALALSLIASVAAAPTATLANGDTITGLNAIINE | |
| | | AFLGIPFAEPPVGNLRFKDPVPYSGSLDGQKFTSYG | |
| | | PSCMQQNPEGTYEENLPKAALDLVMQSKVFEAVSPS | |
| | | SEDCLTINVVRPPGTKAGANLPVMLWIFGGGFEVGG | |
| | | TSTFPPAQMITKSIAMGKPIIHVSVNYRVSSWGFLA | |
| | | GDEIKAEGSANAGLKDQRLGMQWVADNIAAFGGDPT | |
| | | KVTIFGESAGSMSVMCHILWNDGDNTYKGKPLFRAG | |
| | | IMQSGAMVPSDAVDGIYGNEIFDLLASNAGCGSASD | |
| | | KLACLRGVSSDTLEDATNNTPGFLAYSSLRLSYLPR | |
| | | PDGVNITDDMYALVREGKYANIPVIIGDQNDEGTFF | |
| | | GTSSLNVTTDAQAREYFKQSFVHASDAEIDTLMTAY | |
| | | PGDITQGSPFDTGILNALTPQFKRISAVLGDLGFTL | |
| | | ARRYFLNHYTGGTKYSFLSKQLSGLPVLGTFHSNDI | |
| | | VFQDYLLGSGSLIYNNAFIAFATDLDPNTAGLLVKW | |
| | | PEYTSSSQSGNNLMMINALGLYTGKDNFRTAGYDAL | |
| | | FSNPPSFFV | |
| Lipase 2 | Diutina rugosa | >sp\|P32946\|LIP2_DIURU Lipase 2 | 3 |
| | (Candida | OS = Diutina rugosa | |
| | rugosa) | GN = LIP2 PE = 1 SV = 1 | |
| | | MKLCLLALGAAVAAAPTATLANGDTITGLNAIVNEK | |
| | | FLGIPFAEPPVGTLRFKPPVPYSASLNGQQFTSYGP | |
| | | SCMQMNPMGSFEDTLPKNARHLVLQSKIFQVVLPND | |
| | | EDCLTINVIRPPGTRASAGLPVMLWIFGGGFELGGS | |
| | | SLFPGDQMVAKSVLMGKPVIHVSMNYRVASWGFLAG | |
| | | PDIQNEGSGNAGLHDQRLAMQWVADNIAGFGGDPSK | |
| | | VTIYGESAGSMSTFVHLVWNDGDNTYNGKPLFRAAI | |
| | | MQSGCMVPSDPVDGTYGTEIYNQVVASAGCGSASDK | |
| | | LACLRGLSQDTLYQATSDTPGVLAYPSLRLSYLPRP | |
| | | DGTFITDDMYALVRDGKYAHVPVIIGDQNDEGTLFG | |
| | | LSSLNVTTDAQARAYFKQSFIHASDAEIDTLMAAYT | |
| | | SDITQGSPFDTGIFNAITPQFKRISALLGDLAFTLA | |
| | | RRYFLNYYQGGTKYSFLSKQLSGLPVLGTFHGNDII | |
| | | WQDYLVGSGSVIYNNAFIAFANDLDPNKAGLWTNWP | |
| | | TYTSSSQSGNNLMQINGLGLYTGKDNFRPDAYSALF | |
| | | SNPPSFFV | |
| Lipase 3 | Diutina rugosa | >sp\|P32947\|LIP3_DIURU Lipase 3 | 4 |
| | (Candida | OS = Diutina rugosa | |
| | rugosa) | GN = LIP3 PE = 1 SV = 1 | |
| | | MKLALALSLIASVAAAPTAKLANGDTITGLNAIINE | |
| | | AFLGIPFAEPPVGNLRFKDPVPYSGSLNGQKFTSYG | |
| | | PSCMQQNPEGTFEENLGKTALDLVMQSKVFQAVLPQ | |
| | | SEDCLTINVVRPPGTKAGANLPVMLWIFGGGFEIGSP | |
| | | TIFPPAQMVTKSVLMGKPIIHVAVNYRVASWGFLAGD | |
| | | DIKAEGSGNAGLKDQRLGMQWVADNIAGFGGDPSKVT | |
| | | IFGESAGSMSVLCHLIWNDGDNTYKGKPLFRAGIMQ | |
| | | SGAMVPSDPVDGTYGNEIYDLFVSSAGCGSASDKLA | |
| | | CLRSASSDTLLDATNNTPGFLAYSSLRLSYLPRPDG | |
| | | KNITDDMYKLVRDGKYASVPVIIGDQNDEGTIFGLS | |
| | | SLNVTTNAQARAYFKQSFIHASDAEIDTLMAAYPQD | |
| | | ITQGSPFDTGIFNAITPQFKRISAVLGDLAFIHARR | |
| | | YFLNHFQGGTKYSFLSKQLSGLPIMGTFHANDIVWQ | |
| | | DYLLGSGSVIYNNAFIAFATDLDPNTAGLLVNWPKY | |
| | | TSSSQSGNNLMMINALGLYTGKDNFRTAGYDALMTN | |
| | | PSSFFV | |
| Lipase 4 | Diutina rugosa | >sp\|P32948\|LIP4_DIURU Lipase 4 | 5 |
| | (Candida | OS = Diutina rugosa | |
| | rugosa) | GN = LIP4 PE = 3 SV = 1 | |
| | | MKLALVLSLIVSVAAAPTATLANGDTITGLNAIINEA | |
| | | FLGIPFAQPPVGNLRFKPPVPYSASLNGQKFTSYGPS | |
| | | CMQMNPLGNWDSSLPKAAINSLMQSKLFQAVLPNGED | |
| | | CLTINVVRPSGTKPGANLPVMVWIFGGGFEVGGSSLF | |
| | | PPAQMITASVLMGKPIIHVSMNYRVASWGFLAGPDIK | |
| | | AEGSGNAGLHDQRLGLQWVADNIAGFGGDPSKVTIFG | |
| | | ESAGSMSVMCQLLWNDGDNTYNGKPLFRAAIMQSGAM | |
| | | VPSDPVDGPYGTQIYDQVVASAGCGSASDKLACLRSI | |
| | | SNDKLFQATSDTPGALAYPSLRLSFLPRPDGTFITDD | |
| | | MFKLVRDGKCANVPVIIGDQNDEGTVFALSSLNVTTD | |
| | | AQARQYFKESFIHASDAEIDTLMAAYPSDITQGSPFD | |
| | | TGIFNAITPQFKRIAAVLGDLAFTLPRRYFLNHFQGG | |
| | | TKYSFLSKQLSGLPVIGTHHANDIVWQDFLVSHSSAV | |
| | | YNNAFIAFANDLDPNKAGLLVNWPKYTSSSQSGNNLL | |
| | | QINALGLYTGKDNFRTAGYDALFTNPSSFFV | |
| Lipase 5 | Diutina rugosa | >sp\|P32949\|LIP5_DIURU Lipase 5 | 6 |
| | (Candida | OS = Diutina rugosa | |
| | rugosa) | GN = LIP5 PE = 3 SV = 1 | |
| | | MKLALALSLIASVAAAPTATLANGDTITGLNAIINEA | |
| | | FLGIPFAEPPVGNLRFKDPVPYRGSLNGQSFTAYGPS | |
| | | CMQQNPEGTYEENLPKVALDLVMQSKVFQAVLPNSED | |
| | | CLTINVVRPPGTKAGANLPVMLWIFGGGFEIGSPTIF | |
| | | PPAQMVSKSVLMGKPIIHVAVNYRLASFGFLAGPDIK | |
| | | AEGSSNAGLKDQRLGMQWVADNIAGFGGDPSKVTIFG | |
| | | ESAGSMSVLCHLLWNGGDNTYKGKPLFRAGIMQSGAM | |
| | | VPSDPVDGTYGTQIYDTLVASTGCSSASNKLACLRGL | |
| | | STQALLDATNDTPGFLSYTSLRLSYLPRPDGANITDD | |
| | | MYKLVRDGKYASVPVIIGDQNDEGFLFGLSSLNTTTE | |
| | | ADAEAYLRKSFIHATDADITALKAAYPSDVTQGSPFD | |
| | | TGILNALTPQLKRINAVLGDLTFTLSRRYFLNHYTGG | |
| | | PKYSFLSKQLSGLPILGTFHANDIVWQHFLLGSGSVI | |
| | | YNNAFIAFATDLDPNTAGLSVQWPKSTSSSQAGDNLM | |
| | | QISALGLYTGKDNFRTAGYNALPHADPSHFFV | |
| Lipase | Rhizopus niveus | >sp\|P61871\|LIP_RHINI Lipase | 7 |
| | | OS = Rhizopus niveus PE = 1 | |
| | | SV = 1 | |
| | | MVSFISISQGVSLCLLVSSMMLGSSAVPVSGKSGSSN | |
| | | TAVSASDNAALPPLISSRCAPPSNKGSKSDLQAEPYN | |
| | | MQKNTEWYESHGGNLTSIGKRDDNLVGGMTLDLPS | |
| | | DAPPISLSSSTNSASDGGKVVAATTAQIQEFTKYAGI | |
| | | AATAYCRSVVPGNKWDCVQCQKWVPDGKIITTFTSLL | |
| | | SDTNGYVLRSDKQKTIYLVFRGTNSFRSAITDIVFNF | |
| | | SDYKPVKGAKVHAGFLSSYEQVVNDYFPVVQEQLTAH | |
| | | PTYKVIVTGHSLGGAQALLAGMDLYQREPRLSPKNLS | |
| | | IFTVGGPRVGNPTFAYYVESTGIPFQRTVHKRDIVPH | |
| | | VPPQSFGFLHPGVESWIKSGTSNVQICTSEIETKDCS | |
| | | NSIVPFTSILDHLSYFDINEGSCL | |
| Lipase | Aspergillus | >tr\|G3XZX5\|G3XZX5_ASPNA Lipase | 8 |
| | niger (strain | OS = Aspergillus niger | |
| | ATCC 1015/ | (strain ATCC 1015/CBS 113.46/ | |
| | CBS 113.46/ | FGSC A1144/LSHB Ac4/ | |
| | FGSC A1144/ | NCTC 3858a/NRRL 328/USDA 3528.7) | |
| | LSHB Ac4/ | GN = ASPNIDRAFT_53361 PE = 4 SV = 1 | |
| | NCTC 3858a/ | MYIPSVLLLAASLFHGATALPTPGSTPIPPSQDPWYS | |
| | NRRL 328/ | APEGFEEADPGAILRVRPAPGNLTVVVGNASAAYNIL | |
| | USDA 3528.7) | YRTTDSQYKPSWAVTTLLVPPVAASAAVNQSVLLSYQ | |
| | | IAYDSFDVNASPSYAMYTSPPSDIILALQRGWFVNVP | |
| | | DYEGPNASFTAGVQSGHATLDSVRSVLASGFGLNEDA | |
| | | QYALWGYSGGALASEWAAELQMQYAPELNIAGLAVGG | |
| | | LTPNVTSVMDTVTSTISAGLIPAAALGLSSQHPETYE | |
| | | FILSQLKTTGPYNRTGFLAAKDLTLSEAEVFYAFQNI | |
| | | FDYFVNGSATFQAEVVQKALNQDGYMGYHGFPQMPVL | |
| | | AYKAIHDEISPIQDTDRVIKRYCGLGLNILYERNTIG | |
| | | GHSAEQVNGNARAWNWLTSIFDGTYAQQYKTEGCTIR | |
| | | NVTLNTTSSVY | |
| Lipase | Penicillium | >tr\|A0A0G4PG74\|A0A0G4PG74_PENCA | 9 |
| | camemberti | Lipase, GDSL | |
| | FM013 | OS = Penicillium camemberti FM 013 | |
| | | GN = PCAMFM013_S014g000212 PE = 4 | |
| | | SV = 1 | |
| | | MATIETQGNEDAFKPYDQFLLFGDSITQMACNQELG | |
| | | FAFHAGLQESYSRRLDVINRGLAGYSTAHAVKVFDK | |
| | | FFPSPQTANVRFMTIFFGANDACVPTHNQHVPLDQY | |
| | | KENLKTIIQHPATRAQNPRLILISPPPVNEHQLEAF | |
| | | DAAKDTPFPSRTASFTKSYAVAACEVGASLNIPVVD | |
| | | LWSAFMKPTGWKEGEPLIGARDVPSNDTLASLLTDG | |
| | | LHLTPAGNRIVYDELMKVIQANWPDQTPEVLPMVFP | |
| | | SWGDAPK | |

It is preferable to employ lipase B from Candida antarctica of SEQ ID no. 1 or a homologous enzyme having a sequence identity of at least 65% with the sequence of SEQ ID no. 1 and having the same function as the lipase B from Candida antarctica of SEQ ID no. 1.

Step ii) employs a lipase of any of SEQ ID nos. 1 to 9 or a homologous enzyme having a sequence identity of at least 65% with one of the sequences of SEQ ID no. 1 to SEQ ID no. 9 and having the same function as the lipase of SEQ ID no. 1 to SEQ ID no. 9. It is preferable when the homologous enzyme has a sequence identity of at least 80%, preferably of at least 90%, more preferably of at least 95%, more preferably of at least 98%, with the sequence of SEQ ID no. 1 to SEQ ID no. 9 and the same function as the lipase.

In the bile acid derivative of general formula I/II according to the invention at least one of the rings B and D has a further, preferably alpha-, hydroxyl group. The ring B preferably has one or two further, preferably alpha-, hydroxyl group(s) (positions 6 and/or 7). The C-ring of the bile acid derivative of general formula I/II more preferably has no further hydroxyl group (position 12 and/or 13), more preferably none of the rings A, C and D has further hydroxyl groups (positions 1, 2, 12, 13, 15, 16). In a preferred embodiment the ring B therefore has one or two further, preferably alpha-, hydroxyl group(s) (positions 6 and/or 7) and none of the rings A, C and D has further hydroxyl groups (positions 1, 2, 12, 13, 15, 16) save for the 3-alpha-OH group on ring A of the bile acid derivative of general formula I/the alpha-R²—O group on the A-ring of the bile acid derivative of general formula II.

In a preferred embodiment the ring B has one or two further, preferably alpha-, hydroxyl group(s) at position 6 or at positions 6 and 7 respectively and none of the rings A, C and D has further hydroxyl groups (positions 1, 2, 12, 13, 15, 16) save for the 3-alpha-OH group on ring A of the bile acid derivative of general formula I/the alpha-R²—O group on the A-ring of the bile acid derivative of general formula II.

In a preferred embodiment the bile acid derivative of general formula I is selected from the group consisting of R¹ esters of chenodeoxycholic acid (CDCA), hyodeoxycholic acid (HDCA) and hyocholic acid (HCA) and mixtures of two or more thereof, wherein R¹ is as defined hereinabove for general formula I/II. It has been found that, surprisingly, when using a lipase selected from the above recited group, preferably from lipase B from Candida antarctica of SEQ ID no. 1 or a homologous enzyme having a sequence identity of at least 65% with the sequence of SEQ ID no. 1 and having the same function as the lipase B from Candida antarctica of SEQ ID no. 1, more preferably when using lipase B from Candida antarctica of SEQ ID no. 1, both hyodeoxycholic acid (HDCA) and hyocholic acid (HCA) are converted such that only the 3-alpha-hydroxyl group on the A-ring reacted despite the presence of an alpha-hydroxyl group in position 6 on the B-ring/of hydroxyl groups in positions 6 and 7 on the B-ring which would at least co-react under other acylation conditions.

In a further preferred embodiment the bile acid derivative of general formula I is therefore selected from the group consisting of R1 esters of hyodeoxycholic acid (HDCA, 3α,6α-dihydroxycholanic acid), hyocholic acid (HCA, 3α,6α,7α-trihydroxy-5β-cholan-24-oic acid) and mixtures of R¹ esters of HDCA and HCA, wherein R¹ is as defined hereinabove for general formula I/II.

The invention further relates to a bile acid derivative of general formula II obtained or obtainable by a process as described above.

The invention further relates to a bile acid derivative of general formula II,

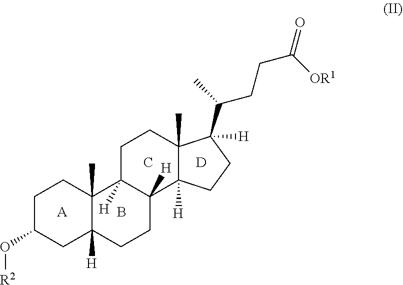

wherein the radical R¹ is selected from the group consisting of C1- to C30-alkyl group, C1- to C30-alkenyl group, C1- to C30-alkynyl group, C5- to C12-cycloalkyl group and C5- to C12-aryl group; and the radical R² is a —C(═O)—C1- to C30-alkyl group; and at least one of the rings B and D has at least one further, preferably alpha-, hydroxyl group (positions 6, 7, 15, 16). The bile acid derivative of general formula II preferably has the formula IIa, IIb or IIc, more preferably the formula IIb or IIc:

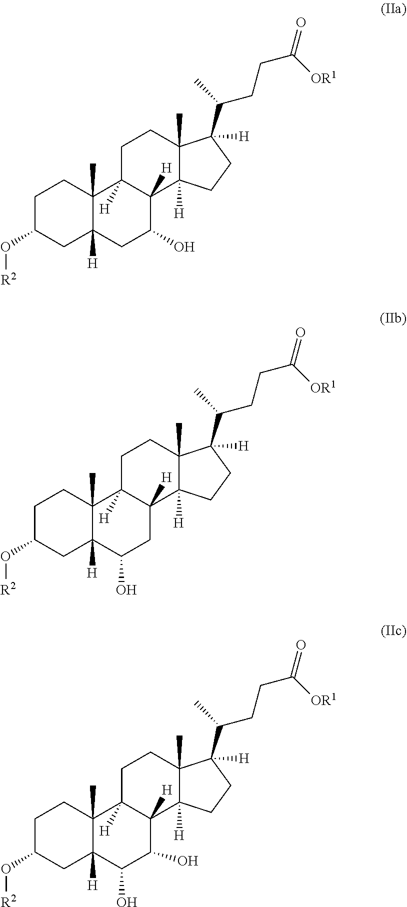

wherein the radical R¹ is in each case selected from the group consisting of C1- to C30-alkyl group, C1- to C30-alkenyl group, C1- to C30-alkynyl group, C5- to C12-cycloalkyl group and C5- to C12-aryl group, preferably from the group consisting of C1- to C18-alkyl group, C5- to C7-cycloalkyl group and phenyl group, more preferably of C1- to C5-alkyl group, more preferably is an unbranched C1- to C3-alkyl radical, more preferably a methyl radical; and the radical R² is a —C(═O)—C1- to C30-alkyl group, preferably an unbranched —C(═O)—C1- to C18-alkyl group, more preferably an unbranched —C(═O)—C1- to C5-alkyl group, more preferably —C(═O)—CH₃.

In a preferred embodiment the invention therefore relates to a bile acid derivative of formula IIa, wherein the radical R¹ is a methyl radical and the radical R² is a —C(═O)—CH₃ (alpha-3′-acetyl-chenodeoxycholic acid methyl ester). In a further preferred embodiment the invention relates to a bile acid derivative of formula IIb, wherein the radical R¹ is a methyl radical and the radical R² is a —C(═O)—CH₃ (alpha-3′-acetyl-hyodeoxycholic acid methyl ester). In a further preferred embodiment the invention relates to a bile acid derivative of formula IIc, wherein the radical R¹ is a methyl radical and the radical R² is a —C(═O)—CH₃ (alpha-3′-acetyl-hyocholic acid methyl ester). In a particularly preferred embodiment the invention relates to a bile acid derivative of formula IIb, wherein the radical R¹ is a methyl radical and the radical R² is a —C(═O)—CH₃ (alpha-3′-acetyl-hyodeoxycholic acid methyl ester) and/or a bile acid derivative of formula IIc, wherein the radical R¹ is a methyl radical and the radical R² is a —C(═O)—CH₃ (alpha-3′-acetyl-hyocholic acid methyl ester).

The invention further relates to the use of a bile acid derivative of general formula II

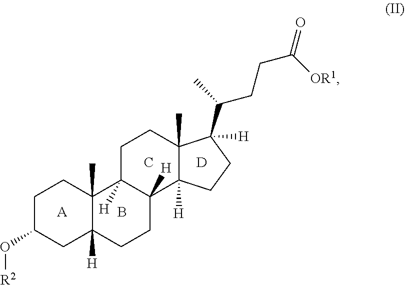

preferably a bile acid derivative of general formula II obtained or obtainable by the process as described above, wherein the radical R¹ is in each case selected from the group consisting of C1- to C30-alkyl group, C1- to C30-alkenyl group, C1- to C30-alkynyl group, C5- to C12-cycloalkyl group and C5- to C12-aryl group, preferably from the group consisting of C1- to C18-alkyl group, C5- to C7-cycloalkyl group and phenyl group, more preferably of C1- to C5-alkyl group, more preferably is an unbranched C1- to C3-alkyl radical, more preferably a methyl radical; and the radical R² is a —C(═O)—C1- to C30-alkyl group, preferably an unbranched —C(═O)—C1- to C18-alkyl group, more preferably an unbranched —C(═O)—C1- to C5-alkyl group, more preferably —C(═O)—CH3; and at least one of the rings B and D has at least one further, preferably alpha, hydroxyl group, for producing lithocholic acid. Preferably employed for producing lithocholic acid is a bile acid derivative of general formula II, wherein at least one of the rings B and D has at least one further, preferably alpha, hydroxyl group. The ring B preferably has one or two further, preferably alpha-, hydroxyl group(s) (positions 6 and/or 7). The C-ring of the bile acid derivative of general formula II more preferably has no further hydroxyl group (position 12 and/or 13), more preferably none of the rings A, C and D has further hydroxyl groups (positions 1, 2, 12, 13, 15, 16). In a preferred embodiment the ring B therefore has one or two further, preferably alpha-, hydroxyl group(s) (positions 6 and/or 7) and none of the rings A, C and D has further hydroxyl groups (positions 1, 2, 12, 13, 15, 16) save for the 3-alpha-R²—O group on ring A of the bile acid derivative of general formula II.

In a preferred embodiment the bile acid derivative of general formula II which is used for producing lithocholic acid is selected from the group consisting of R¹-, R²-derivatives of chenodeoxycholic acid (CDCA), hyodeoxycholic acid (HDCA), hyocholic acid (HCA) and mixtures of two or more thereof, wherein R¹ and R² are as defined hereinabove. In a particularly preferred embodiment the bile acid derivative of general formula II which is used for producing lithocholic acid is selected from the group consisting of R¹-, R²-derivatives of hyodeoxycholic acid (HDCA), hyocholic acid (HCA) and mixtures of R¹-, R²-derivatives of hyodeoxycholic acid (HDCA) and R¹-, R²-derivatives of hyocholic acid (HCA), wherein R¹ and R² are as defined hereinabove.

The bile acid derivative of general formula II which is used for producing lithocholic acid therefore preferably has the formula IIa, IIb or IIc, more preferably the formula IIb or IIc:

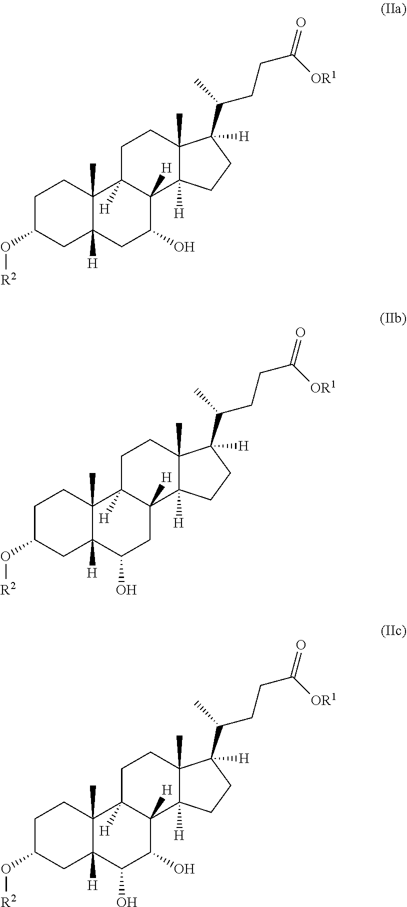

wherein the radical R¹ is in each case selected from the group consisting of C1- to C30-alkyl group, C1- to C30-alkenyl group, C1- to C30-alkynyl group, C5- to C12-cycloalkyl group and C5- to C12-aryl group, preferably from the group consisting of C1- to C18-alkyl group, C5- to C7-cycloalkyl group and phenyl group, more preferably of C1- to C5-alkyl group, more preferably is an unbranched C1- to C3-alkyl radical, more preferably a methyl radical; and the radical R² is a —C(═O)—C1- to C30-alkyl group, preferably an unbranched —C(═O)—C1- to C18-alkyl group, more preferably an unbranched —C(═O)—C1- to C5-alkyl group, more preferably —C(═O)—CH₃.

In a preferred embodiment the invention therefore relates to the use of a bile acid derivative of formula IIa, wherein the radical R¹ is a methyl radical and the radical R² is a —C(═O)—CH₃, for producing lithocholic acid. In a further preferred embodiment the invention therefore relates to the use of a bile acid derivative of formula IIb, wherein the radical R¹ is a methyl radical and the radical R² is a —C(═O)—CH3, for producing lithocholic acid. In a further preferred embodiment the invention therefore relates to the use of a bile acid derivative of formula IIc, wherein the radical R¹ is a methyl radical and the radical R² is a —C(═O)—CH₃, for producing lithocholic acid. In a particularly preferred embodiment the invention therefore relates to the use of a bile acid derivative of formula IIb, wherein the radical R¹ is a methyl radical and the radical R² is a —C(═O)—CH₃ for producing lithocholic acid and/or the use of a bile acid derivative of formula IIc, wherein the radical R¹ is a methyl radical and the radical R² is a —C(═O)—CH₃, for producing lithocholic acid.

Production of lithocholic acid may be carried out using a single bile acid derivative of general formula II or a mixture of two or more bile acid derivatives of general formula II. In a preferred embodiment a mixture of the bile acid derivatives of formula IIa, IIb and IIc, more preferably a mixture of the bile acid derivatives of formula IIb and IIc, is used.

The invention further relates to a process for producing lithocholic acid, comprising
i) providing a first composition comprising at least one bile acid derivative of general formula I:

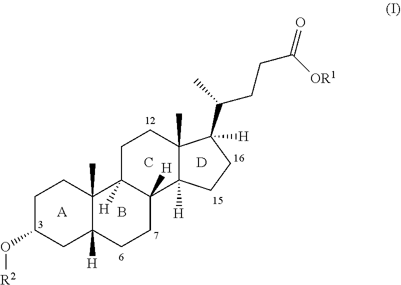

wherein the radical R¹ is selected from the group consisting of C1- to C30-alkyl group, C1- to C30-alkenyl group, C1- to C30-alkynyl group, C5- to C12-cycloalkyl group and C5- to C12-aryl group and at least one of the rings B and D has at least one further, preferably alpha-, hydroxyl group (positions 6, 7, 15, 16);
ii) contacting the first composition comprising at least one bile acid derivative of general formula I from i) with a compound R²—X, wherein R² is a —C(═O)—C1- to C30-alkyl group and X is selected from the group consisting of hydroxyl group, —O—C1- to C20-alkyl group, —O—C1- to C20-alkenyl group, —O—C1- to C20-alkynyl group, thiol group, —S—C1- to C20-alkyl group, amine group, —NHR³ group, —NR³R⁴ group, wherein R³ and R⁴ are each independently a C1- to C20-alkyl group, halogen atom and —O—(C═O)—R⁵ group, wherein R⁵ is a C1- to C20-alkyl group; and a lipase selected from the group consisting of SEQ ID no. 1 to SEQ ID no. 9 or a homologous enzyme having a sequence identity of at least 65% with the sequence of SEQ ID no. 1 to SEQ ID no. 9 and having the same function as the lipase of SEQ ID no. 1 to SEQ ID no. 9 to obtain a second composition comprising at least one bile acid derivative of general formula II:

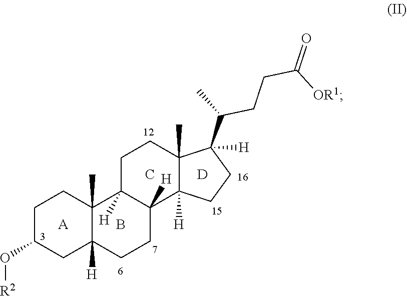

wherein the radical R¹ is as defined at i) for formula I and the radical R² is as defined at ii) and at least one of the rings B and D has at least one further, preferably alpha-, hydroxyl group;
iii) conversion of the at least one bile acid derivative of general formula II obtained from ii) into lithocholic acid.

The bile acid derivative obtained from ii) and reacted in iii) has the general formula II

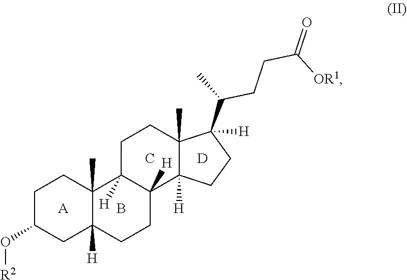

wherein the radical R¹ is in each case selected from the group consisting of C1- to C30-alkyl group, C1- to C30-alkenyl group, C1- to C30-alkynyl group, C5- to C12-cycloalkyl group and C5- to C12-aryl group, preferably from the group consisting of C1- to C18-alkyl group, C5- to C7-cycloalkyl group and phenyl group, more preferably of C1- to C5-alkyl group, more preferably is an unbranched C1- to C3-alkyl radical, more preferably a methyl radical; and the radical R² is a branched or unbranched-C(═O)—C1- to C30-alkyl group, preferably an unbranched —C(═O)—C1- to C18-alkyl group, more preferably an unbranched —C(═O)—C1- to C5-alkyl group, more preferably —C(═O)—CH3; and at least one of the rings B and D has at least one further, preferably alpha-, hydroxyl group. It is preferable when in the bile acid derivative of general formula II obtained in ii) and converted in iii) at least one of the rings B and D has at least one further, preferably alpha-, hydroxyl group. The ring B preferably has one or two further, preferably alpha-, hydroxyl group(s) (positions 6 and/or 7). The C-ring of the bile acid derivative of general formula II more preferably has no further hydroxyl group (position 12 and/or 13), more preferably none of the rings A, C and D has further hydroxyl groups (positions 1, 2, 12, 13, 15, 16). In a preferred embodiment the ring B therefore has one or two further, preferably alpha-, hydroxyl group(s) (positions 6 and/or 7) and none of the rings A, C and D has further hydroxyl groups (positions 1, 2, 12, 13, 15, 16) save for the 3-alpha-OH group on ring A of the bile acid derivative of general formula II. In a more preferred embodiment the ring B has one or two further, preferably alpha-, hydroxyl group(s) (position 6 or positions 6 and 7) and none of the rings A, C and D has further hydroxyl groups (positions 1, 2, 12, 13, 15, 16) save for the 3-alpha-OH group on ring A of the bile acid derivative of general formula II.

In a preferred embodiment the bile acid derivative of general formula II obtained in ii) and converted in iii) is selected from the group consisting of R¹-, R²-derivatives of chenodeoxycholic acid (CDCA), hyodeoxycholic acid (HDCA), hyocholic acid (HCA) and mixtures of two or more thereof, wherein R¹ and R² are as defined hereinabove. In a further preferred embodiment the bile acid derivative of general formula II obtained in ii) and converted in iii) is selected from the group consisting of R¹-, R²-derivatives of hyodeoxycholic acid (HDCA), hyocholic acid (HCA) and mixtures of R¹-, R²-derivatives of hyodeoxycholic acid (HDCA) and R¹-, R²-derivatives of hyocholic acid (HCA), wherein R¹ and R² are as defined hereinabove.

The bile acid derivative of general formula II obtained in ii) and converted in iii) therefore preferably has the formula IIa, IIb or IIc, more preferably the formula IIb or IIc:

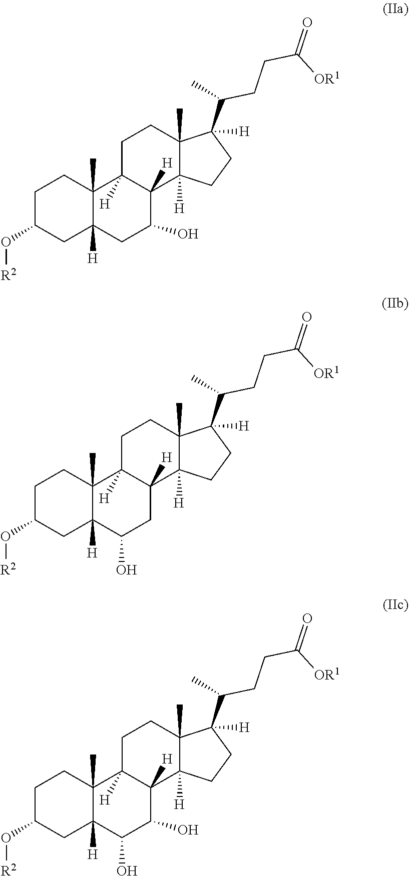

wherein the radical R¹ is in each case selected from the group consisting of C1- to C30-alkyl group, C1- to C30-alkenyl group, C1- to C30-alkynyl group, C5- to C12-cycloalkyl group and C5- to C12-aryl group, preferably from the group consisting of C1- to C18-alkyl group, C5- to C7-cycloalkyl group and phenyl group, more preferably of C1- to C5-alkyl group, more preferably is an unbranched C1- to C3-alkyl radical, more preferably a methyl radical; and the radical R² is a branched or unbranched —C(═O)—C1- to C30-alkyl group, preferably an unbranched —C(═O)—C1- to C18-alkyl group, more preferably an unbranched —C(═O)—C1- to C5-alkyl group, more preferably —C(═O)—CH3.

In a preferred embodiment in the bile acid derivative of formula IIa the radical R¹ is a methyl radical and the radical R² is a —C(═O)—CH3. In a further preferred embodiment in the bile acid derivative of formula IIb the radical R¹ is a methyl radical and the radical R² is a —C(═O)—CH₃. In a further preferred embodiment in the bile acid derivative of formula IIc the radical is R¹ is a methyl radical and the radical R² is a —C(═O)—CH₃.

In a preferred embodiment in the process for producing lithocholic acid a single bile acid derivative of general formula II or a mixture of two or more bile acid derivatives of general formula II are employed. In a particularly preferred embodiment a mixture of the bile acid derivatives of formulae IIa, IIb and IIc, more preferably a mixture of the bile acid derivatives of formulae IIb and IIc, are employed.

The invention further relates to a process for producing lithocholic acid, comprising
a) providing a composition comprising a bile acid derivative of general formula II, preferably obtained or obtainable by the process as described above,

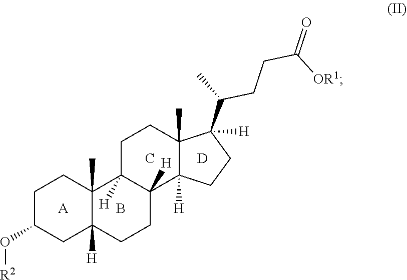

wherein the radical R¹ is selected from the group consisting of C1- to C30-alkyl group, C1- to C30-alkenyl group, C1- to C30-alkynyl group, C5- to C12-cycloalkyl group and C5- to C12-aryl group; the radical R² is a —C(═O)—C1- to C30-alkyl group; and at least one of the rings B and D has at least one further, preferably alpha-, hydroxyl group;
b) contacting the composition comprising a bile acid derivative of general formula II from a) with an oxidant or a C1- to C10-alkylthiol, preferably propanethiol, to convert the at least one hydroxyl group in B and/or D into an ═O group or an —S—C1- to C10-alkyl group, preferably an ═O group or an —S-propyl group to obtain a bile acid derivative of general formula III,

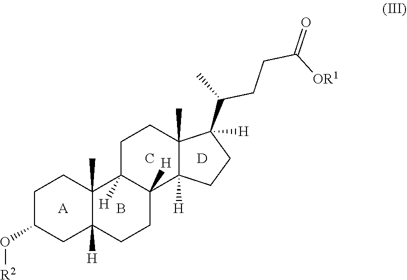

wherein the radical R¹ and the radical R² are as defined in general formula II and at least one of the rings B and D has at least one ═O group or an —S—C1- to C10-alkyl group, preferably an ═O group or an —S-propyl group;
c) contacting the bile acid derivative of general formula III from b) with a reducing agent, optionally with additional saponification, to obtain lithocholic acid.

The oxidant employed in b) preferably comprises one or more compounds selected from the group consisting of pyridinium chlorochromate (PCC), hypochlorite, hypobromite, dichromate, chromic acid, Dess-Martin periodane (1,1,1-triacetoxy-1,1-dihydro-1,2-benziodoxol-3(1H)-one), oxalylchloride/DMSO, hydrogen peroxide, oxygen, iodine, potassium permanganate, C1- to C30-peracids, percarbonate, potassium peroxomonosulfate and dimethylchlorosulphonium ion, preferably from the group consisting of pyridinium chlorochromate (PCC), hypochlorite, hypobromite, dichromate, chromic acid, hydrogen peroxide, potassium permanganate, C1- to C30-peracids and percarbonate, more preferably hypochlorite or hypobromite, more preferably hypochlorite. The reducing agent used in c) preferably comprises one or more compounds selected from the group consisting of hydrazine, hydrazine derivative, preferably tosylhydrazine, semicarbazide; hydrazine hydrate, hydrogen, sodium cyanoborohydride, diisobutylaluminum hyride, lithium aluminum hydride, silane, butyltin hydride, zinc/hydrochloric acid, lithium, sodium and sodium borohydride, more preferably selected from the group consisting of hydrazine, hydrazine derivative, preferably tosylhydrazine, semicarbazide; hydrazine hydrate, hydrogen and sodium borohydride, more preferably hydrazine or sodium borohydride. The reducing agent reduces at least one ═O group or —S—C1- to C10-alkyl group to a methylene group and preferably reliberates the 3-alpha-hydroxyl group on ring A (elimination of the R² group). The optional saponification/the agents and conditions suitable therefor are known to those skilled in the art.

The bile acid derivative present in the composition provided in a) has the general formula II

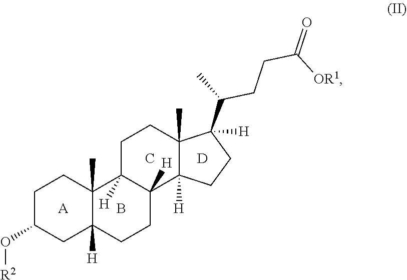

wherein the radical R¹ is in each case selected from the group consisting of C1- to C30-alkyl group, C1- to C30-alkenyl group, C1- to C30-alkynyl group, C5- to C12-cycloalkyl group and C5- to C12-aryl group, preferably from the group consisting of C1- to C18-alkyl group, C5- to C7-cycloalkyl group and phenyl group, more preferably of C1- to C5-alkyl group, more preferably is an unbranched C1- to C3-alkyl radical, more preferably a methyl radical; and the radical R² is a branched or unbranched-C(═O)—C1- to C30-alkyl group, preferably an unbranched —C(═O)—C1- to C18-alkyl group, more preferably an unbranched —C(═O)—C1- to C5-alkyl group, more preferably —C(═O)—CH3; and at least one of the rings B and D has at least one further, preferably alpha-, hydroxyl group. It is preferable when in the bile acid derivative of general formula II present in the composition provided in a) at least one of the rings B and D has at least one further, preferably alpha-, hydroxyl group. The ring B preferably has one or two further, preferably alpha-, hydroxyl group(s) (positions 6 and/or 7). The C-ring of the bile acid derivative of general formula II more preferably has no further hydroxyl group (position 12 and/or 13), more preferably none of the rings A, C and D has further hydroxyl groups (positions 1, 2, 12, 13, 15, 16). In a preferred embodiment the ring B therefore has one or two further, preferably alpha-, hydroxyl group(s) (positions 6 and/or 7) and none of the rings A, C and D has further hydroxyl groups (positions 1, 2, 12, 13, 15, 16) save for the 3-alpha-R²—O group on ring A of the bile acid derivative of general formula II. In a more preferred embodiment the ring B has one or two further, preferably alpha-, hydroxyl group(s) at position 6 or at positions 6 and 7 respectively and none of the rings A, C and D has further hydroxyl groups (positions 1, 2, 12, 13, 15, 16) save for the 3-alpha-R²O group on ring A of the bile acid derivative of general formula II.

In a preferred embodiment the bile acid derivative of general formula II present in the composition provided in a) is selected from the group consisting of R¹-, R²-derivatives of chenodeoxycholic acid (CDCA), hyodeoxycholic acid (HDCA), hyocholic acid (HCA) and mixtures of two or more thereof, wherein R¹ and R² are as defined hereinabove. In a further preferred embodiment the bile acid derivative of general formula II present in the composition provided in a) is selected from the group consisting of R¹-, R²-derivatives of hyodeoxycholic acid (HDCA), hyocholic acid (HCA) and mixtures of R¹-, R²-derivatives of hyodeoxycholic acid (HDCA) and R¹-, R²-derivatives of hyocholic acid (HCA), wherein R¹ and R² are as defined hereinabove.

The bile acid derivative of general formula II present in the composition provided in a) therefore preferably has the formula IIa, IIb or IIc, more preferably the formula IIb or IIc, more preferably the formula IIb:

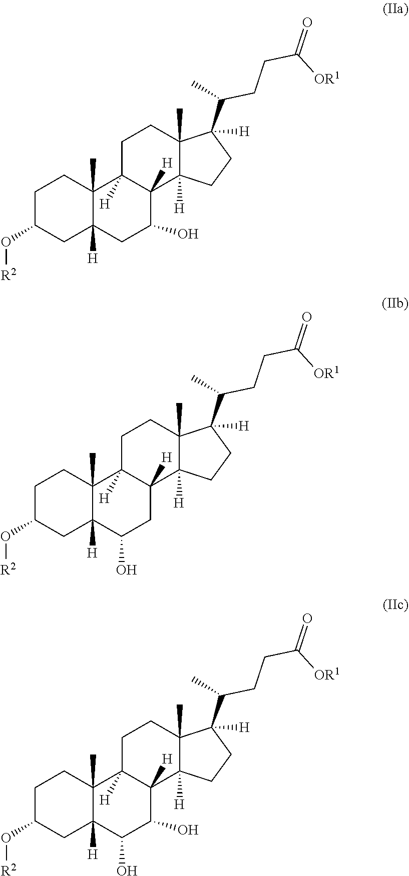

wherein the radical R¹ is in each case selected from the group consisting of C1- to C30-alkyl group, C1- to C30-alkenyl group, C1- to C30-alkynyl group, C5- to C12-cycloalkyl group and C5- to C12-aryl group, preferably from the group consisting of C1- to C18-alkyl group, C5- to C7-cycloalkyl group and phenyl group, more preferably of C1- to C5-alkyl group, more preferably is an unbranched C1- to C3-alkyl radical, more preferably a methyl radical; and the radical R² is a branched or unbranched —C(═O)—C1- to C30-alkyl group, preferably an unbranched —C(═O)—C1- to C18-alkyl group, more preferably an unbranched —C(═O)—C1- to C5-alkyl group, more preferably —C(═O)—CH3.

In a preferred embodiment in the bile acid derivative of formula IIa present in the composition provided in a) the radical R¹ is a methyl radical and the radical R² is a —C(═O)—CH₃. In a further preferred embodiment in the bile acid derivative of formula IIb present in the composition provided in a) the radical R¹ is a methyl radical and the radical R² is —C(═O)—CH₃. In a further preferred embodiment in the bile acid derivative of formula IIc present in the composition provided in a) the radical R¹ is a methyl radical and the radical R² is —C(═O)—CH₃.

A preferred embodiment of the process for producing lithocholic acid comprises:
a) providing a composition comprising a bile acid derivative of general formula IIb, preferably obtained or obtainable by the process as described above,

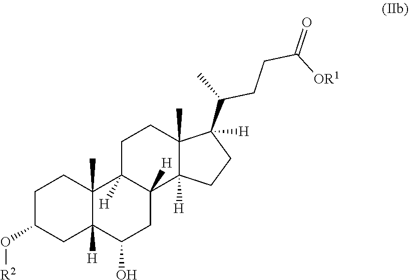

wherein the radical R¹ is selected from the group consisting of C1- to C30-alkyl group, C1- to C30-alkenyl group, C1- to C30-alkynyl group, C5- to C12-cycloalkyl group and C5 to C12-aryl group; the radical R² is a C(═O)—C1- to C30-alkyl group;
b) contacting the composition comprising a bile acid derivative of general formula IIb from a) with an oxidant or a C1- to C10-alkylthiol, preferably propanethiol, to convert the at least one hydroxyl group in B and/or D into an ═O group or an —S—C1- to C10-alkyl group, preferably an ═O group or an —S-propyl group to obtain a bile acid derivative of general formula IIIb,

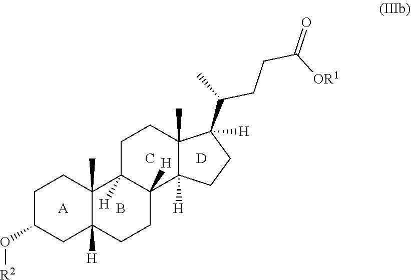

wherein the radical R¹ and the radical R² are as defined in general formula II and the ring B in 6 position has ═O group or an —S—C1- to C10-alkyl group, preferably an ═O group or an —S-propyl group;
c) contacting the bile acid derivative of general formula IIb from b) with a reducing agent, optionally with additional saponification, to obtain lithocholic acid.

The invention further relates to lithocholic acid obtained or obtainable by any of the above-described processes.

The invention further relates to the use of lithocholic acid obtained or obtainable by any of the above-described processes for producing hydroxylated bile acids. The invention relates specifically but not exclusively to the use of lithocholic acid obtained or obtainable by any of the above-described processes for producing ursodeoxycholic acid or ursodeoxycholic acid derivatives.

The present invention is more particularly illustrated by the examples which follow.

### EXAMPLES

### Example 1—Production of Chenodeoxycholic Acid Methyl Ester

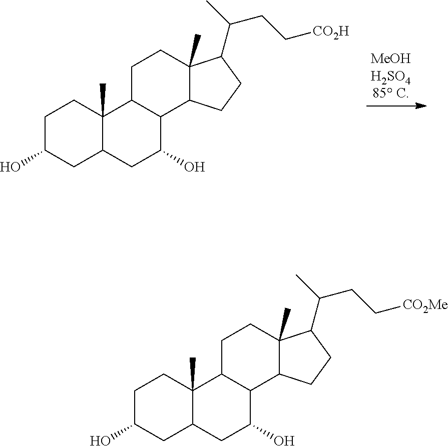 [SMILES: CC(CCC(=O)O)C1CCC2C3C(CC[C@]12C)[C@@]1(C)CC[C@@H](O)CC1C[C@H]3O ; COC(=O)CCC(C)C1CCC2C3C(CC[C@]12C)[C@@]1(C)CC[C@@H](O)CC1C[C@H]3O]

0.5 kg of chenodeoxycholic acid (CDCA) were dissolved in 1.5 L of (technical grade) methanol with stirring in a double-walled glass reactor. 0.0031 L of concentrated sulfuric acid (90%) were added slowly. The temperature was then adjusted to 85° C. and the reaction stirred under reflux. After complete conversion to chenodeoxycholic acid methyl ester (CDCA-Me) the reaction solution was set to 40° C. and 0.75 litres of methanol were distillatively removed under vacuum. 2 litres of ethyl acetate (technical grade) were then added to the solution. The organic phase was washed twice with 1.5 litres of saturated sodium hydrogencarbonate solution and three times with 1.5 litres of saturated sodium chloride solution. The organic phase was subsequently concentrated to dryness under vacuum.

Yield: 0.475 kg of CDCA-Me; 95% based on employed CDCA.

### Example 2—Production of 3′-Acetyl-Chenodeoxycholic Acid Methyl Ester

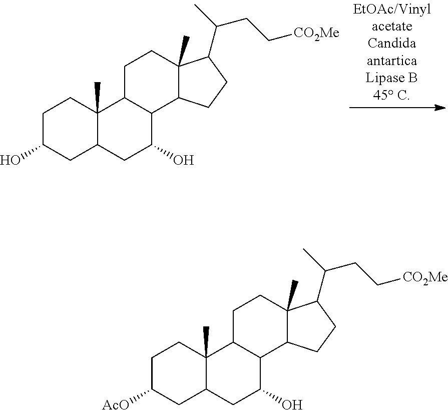 [SMILES: COC(=O)CCC(C)C1CCC2C3C(CC[C@]12C)[C@@]1(C)CC[C@@H](O)CC1C[C@H]3O ; COC(=O)CCC(C)C1CCC2C3C(CC[C@]12C)[C@@]1(C)CC[C@@H](OC(C)=O)CC1C[C@H]3O]

0.475 kg of CDCA-Me from example 1 were dissolved in 1.16 litres of ethyl acetate (technical grade) with stirring in a double-walled glass reactor. Added thereto were 0.25 litres of vinyl acetate (>95%) and 0.0035 kg of immobilized lipase B from Candida antarctica of SEQ ID no. 1. The reaction temperature was set to 45° C. Once the reaction was complete the lipase was filtered off and the solvent concentrated to dryness under vacuum to obtain 3′-acetyl-chenodeoxycholic acid methyl ester (3′-Ac-CDCA-Me) as a solid.

Yield: 0.451 kg of 3′Ac-CDCA-Me; 95% based on employed CDCA-Me.

### Example 3—Production of 3′-Acetyl-7-Oxo-Chenodeoxycholic Acid Methyl Ester

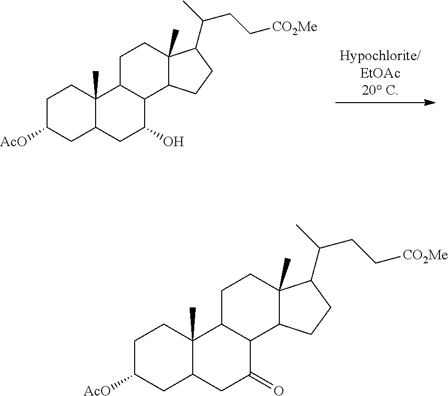 [SMILES: COC(=O)CCC(C)C1CCC2C3C(CC[C@]12C)[C@@]1(C)CC[C@@H](OC(C)=O)CC1C[C@H]3O ; COC(=O)CCC(C)C1CCC2C3C(=O)CC4C[C@H](OC(C)=O)CC[C@]4(C)C3CC[C@]12C]

0.451 kg of 3′Ac-CDCA-Me from example 2 were dissolved in 2.65 litres of ethyl acetate (technical grade) and 0.66 litres of glacial acetic acid. 2.65 litres of sodium hypochlorite solution (5-10% technical grade) were added to the reaction with cooling so that the reaction temperature did not exceed 20° C. Upon complete conversion to the oxo compound the aqueous phase was discharged and the organic phase washed with 0.8 litres of a 10% sodium dithionite solution. The organic phase was washed with 3.5 litres of water and subsequently dried over magnesium sulfate. The dried organic phase was concentrated to dryness under vacuum to obtain 3′-acetyl-7-oxo-chenodeoxycholic acid methyl ester (3′Ac-7-oxo-CDCA-Me) as a solid.

Yield: 0.383 kg of 3′Ac-7-oxo-CDCA-Me; 90% based on employed 3′Ac-CDCA-Me.

### Example 4—Production of Lithocholic Acid

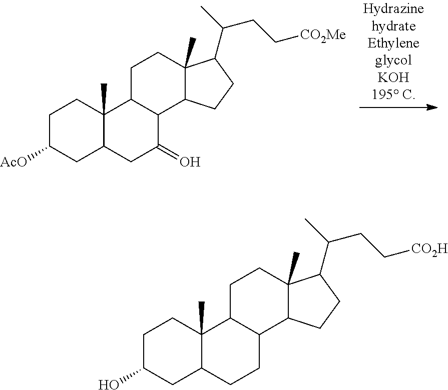 [SMILES: COC(=O)CCC(C)C1CCC2C3C(=O)CC4C[C@H](OC(C)=O)CC[C@]4(C)C3CC[C@]12C ; CC(CCC(=O)O)C1CCC2C3CCC4C[C@H](O)CC[C@]4(C)C3CC[C@]12C]

0.383 kg of 3′-acetyl-7-oxo-chenodeoxycholic acid methyl ester (3′Ac-7-oxo-CDCA-Me) from example 3 were suspended in 1.5 litres of ethylene glycol and 0.425 litres of water were added with stirring. 0.489 kg of solid potassium hydroxide and 4.1 litres of hydrazine hydrate were added to the reaction solution (50% in water). The reaction solution was heated to 130° C. and water and hydrazine hydrate were removed by distillation. Once distillative removal was complete the temperature was set to 195° C. and maintained for 2.5 h. A strong evolution of gas, indicating the progress of the reaction, was observed. The reaction solution was subsequently cooled to below 100° C. and 8.5 litres of a water/ice mixture was then added to the reaction and stirred vigorously. The mixture was then acidified to pH 1 with 0.638 litres of concentrated sulfuric acid. The crude product precipitated as a fine white solid and was filtered off. The crude product was washed with 0.5 litres of water and 0.5 litres of acetonitrile and then dried. The crude lithocholic acid was dissolved in 1.0 L of glacial acetic acid and slowly crystallized by addition of 1.0 litres of water. The produced lithocholic acid was filtered and dried.

Yield: 0.278 kg of lithocholic acid; 90% based on employed 3′Ac-7-oxo-CDCA-Me.

### Example 5—Production of 3′-Acetyl-7-Propylthio-Chenodeoxycholic Acid Methyl Ester

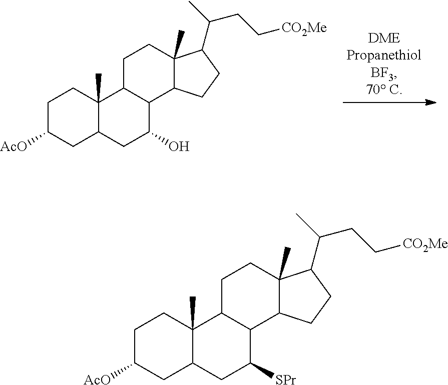

0.451 kg of 3′Ac-CDCA-Me from example 2 were dissolved in 4.5 litres of ethylene glycol is dimethyl ether (DME) and 0.337 litres of propanethiol and 0.135 litres of BF₃×Et₂O were added. The reaction solution was heated under reflux for 2 days. The cooled reaction solution was then washed to neutrality with sodium carbonate solution and the organic phase was concentrated to dryness under vacuum to obtain 3′-acetyl-7-propylthio-chenodeoxycholic acid methyl ester as a solid.

Yield: 0.405 kg of 3′-acetyl-7-propylthio-chenodeoxycholic acid methyl ester; 91% based on employed 3′Ac-CDCA-Me.

### Example 6—Production of Lithocholic Acid

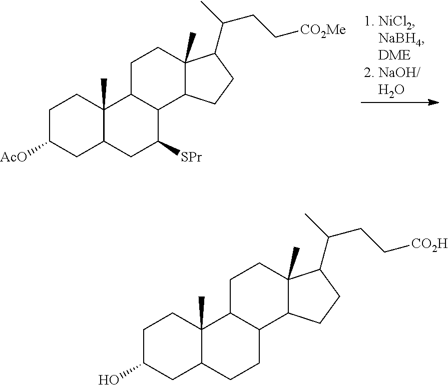

0.405 kg of 3′-acetyl-7-propylthio-chenodeoxycholic acid methyl ester from example 5 and 1.3 kg of nickel chloride hexahydrate were dissolved in 10 litres of methanol-THF (1:1) at 0° C. 0.318 kg of sodium borohydride were added to the reaction solution in small portions of 20 g. Once addition was complete the solution was stirred for a further 30 min. The precipitate was filtered over celite and washed further with methanol-THF. The solvent was removed to dryness under vacuum to obtain crude lithocholic acid as a solid.

The crude lithocholic acid was dissolved in 1.0 L of glacial acetic acid and slowly crystallized by addition of 1.0 litres of water. The produced lithocholic acid was filtered and dried.

Yield: 0.281 kg of lithocholic acid; 70% based on employed 3′-acetyl-7-propylthio-chenodeoxycholic acid methyl ester.

### Example 7—Production of Hyodeoxycholic Acid Methyl Ester

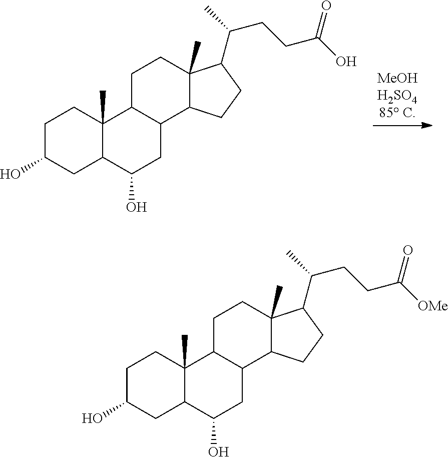 [SMILES: C[C@H](CCC(=O)O)C1CCC2C3C[C@H](O)C4C[C@H](O)CC[C@]4(C)C3CC[C@@]21C ; COC(=O)CC[C@@H](C)C1CCC2C3C[C@H](O)C4C[C@H](O)CC[C@]4(C)C3CC[C@@]21C]

0.5 kg of hyodeoxycholic acid (HDCA) were dissolved in 1.5 L of (technical grade) methanol with stirring in a double-walled glass reactor. 0.0031 L of concentrated sulfuric acid (98%) were added slowly. The temperature was then adjusted to 85° C. and the reaction stirred under reflux. After complete conversion to hyodeoxycholic acid methyl ester (HDCA-Me) the reaction solution was set to 40° C. and 0.75 litres of methanol were distillatively removed under vacuum. 2 Litres of ethyl acetate (technical grade) were then added to the solution. The organic phase was washed twice with 1.5 litres of saturated sodium hydrogencarbonate solution and three times with 1.5 litres of saturated sodium chloride solution. The organic phase was subsequently concentrated to dryness under vacuum.

Yield: 0.485 kg of HDCA-Me; 98% based on employed CDCA.

### Example 8—Production of 3′-Acetyl-Hyodeoxycholic Acid Methyl Ester

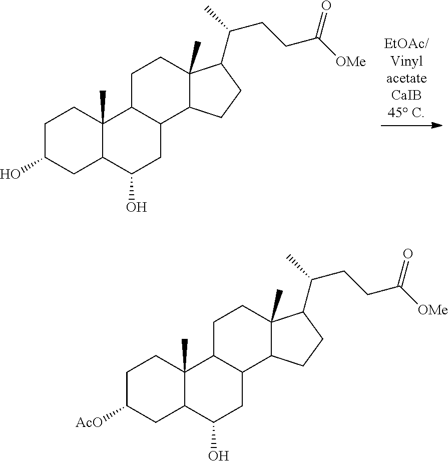 [SMILES: COC(=O)CC[C@@H](C)C1CCC2C3C[C@H](O)C4C[C@H](O)CC[C@]4(C)C3CC[C@@]21C ; COC(=O)CC[C@@H](C)C1CCC2C3C[C@H](O)C4C[C@H](OC(C)=O)CC[C@]4(C)C3CC[C@@]21C]

0.485 kg of HDCA-Me from example 7 were dissolved in 1.16 litres of ethyl acetate (technical grade) with stirring in a double-walled glass reactor. Added thereto were 0.25 litres of vinyl acetate (>95%) and 0.0035 kg of immobilized lipase B from Candida antarctica of SEQ ID no. 1. The reaction temperature was set to 45° C. Once the reaction was complete the lipase was filtered off and the solvent concentrated to dryness under vacuum to obtain 3′-acetyl-hyodeoxycholic acid methyl ester (3′-Ac-HDCA-Me) as a solid.

Yield: 0.470 kg of 3′Ac-HDCA-Me; 96% based on employed HDCA-Me.

It was surprisingly found that in the case of hyodeoxycholic acid (HDCA) only the 3-alpha-hydroxyl group on the A ring was acetylated despite the presence of an alpha-hydroxyl group in position 6 on the B-ring which would at least co-react under other acylation conditions and that the use of lipase B from Candida antarctica of SEQ ID no. 1 resulted in virtually complete acetylation of the 3-alpha-hydroxyl group on the A ring while the 6-alpha-hydroxyl group on the B ring remained as a hydroxyl group.

HCA-Me (0.47 kg) obtained from HCA (0.5 kg) analogously to example 7 was analogously acetylated with vinyl acetate and immobilized lipase B from Candida antarctica of SEQ ID no. 1 (3′Ac-HCA-Me, 0.44 kg, 94%). It was likewise found here that only the 3-alpha-hydroxyl group on the A ring was acetylated despite the presence of alpha-hydroxyl groups in positions 6 and 7 on the B-ring which would at least co-react under other acylation conditions and that the use of lipase B from Candida antarctica of SEQ ID no. 1 resulted in virtually complete acetylation of the 3-alpha-hydroxyl group on the A ring while the 6- and 7-alpha-hydroxyl group on the B-ring remained as hydroxyl groups; these results are clearly apparent from the NMR spectra.

The ¹H- and ¹³C-NMR data are reported in the tables below.

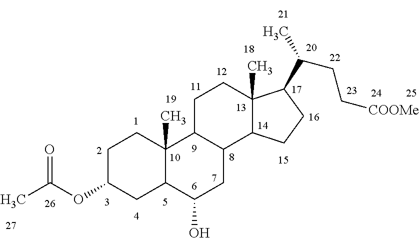 [SMILES: COC(=O)CC[C@@H](C)[C@H]1CCC2C3C[C@H](O)C4C[C@H](OC(C)=O)CC[C@]4(C)C3CC[C@@]21C]

3-Ac-HDCA-Me

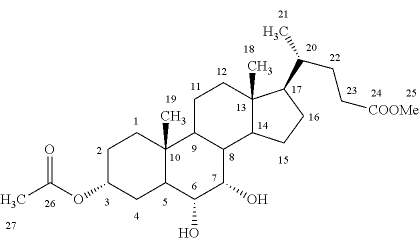 [SMILES: COC(=O)CC[C@@H](C)[C@H]1CCC2C3C(CC[C@@]21C)[C@@]1(C)CC[C@@H](OC(C)=O)CC1[C@@H](O)[C@H]3O]

3-Ac-HCA-Me

| | δ 13C (ppm) | |
|---|---|---|
| No. | 3-Ac-HDCA-Me | 3-Ac-HCA-Me |
| 1 | 35.40 | 35.4 |
| 2 | 26.69 | 26.84 |
| 3 | 74.31 | 74.54 |
| 4 | 25.46 | 28.54 |
| 5 | 48.46 | 47.80 |
| 6 | 67.93 | 69.42 |
| 7 | 34.90 | 72.07 |
| 8 | 34.88 | 38.64 |
| 9 | 39.93 | 32.73 |
| 10 | 36.07 | 36.16 |
| 11 | 20.09 | 20.74 |
| 12 | 40.05 | 39.58 |
| 13 | 42.97 | 42.89 |
| 14 | 56.29 | 50.30 |
| 15 | 24.31 | 23.79 |
| 16 | 28.26 | 28.31 |
| 17 | 56.08 | 55.88 |
| 18 | 12.17 | 11.91 |
| 19 | 23.61 | 23.15 |
| 20 | 35.48 | 35.56 |
| 21 | 18.40 | 18.43 |
| 22 | 31.18 | 31.20 |
| 23 | 31.18 | 31.20 |
| 24 | 174.92 | 175.19 |
| 25 | 51.67 | 51.72 |
| 26 | 170.76 | 171.23 |
| 27 | 21.58 | 21.65 |

δ 1H (ppm)

| No. | 3-Ac-HDCA-Me | 3-Ac-HCA-Me |
|---|---|---|
| 1 | | |
| 2 | | |
| 3 | 4.67, tt | 4.50, tt |
| 4 | | |
| 5 | | |
| 6 | 4.02, dt | 3.80, brs |
| 7 | | 3.83, brt |
| 8 | | |
| 9 | | |
| 10 | | |
| 11 | | |
| 12 | | |
| 13 | | |
| 14 | | |
| 15 | | |
| 16 | | |
| 17 | | |
| 18 | 0.59, s | 0.61 |
| 19 | 0.87, s | 0.88 |
| 20 | | |
| 21 | 0.89, d | 0.89 |
| 22 | | |
| 23 | 2.17, 2.30 | |
| 24 | | |
| 25 | 3.60 | — |
| 26 | | |
| 27 | 1.98 | — |

tt: triplet of triplets; dt: doublet of triplets; s: singlet; d: doublet; brs: broad singlet; brt: broad triplet

### Example 9—Production of 3′-Acetyl-7-Oxo-Hyodeoxycholic Acid Methyl Ester

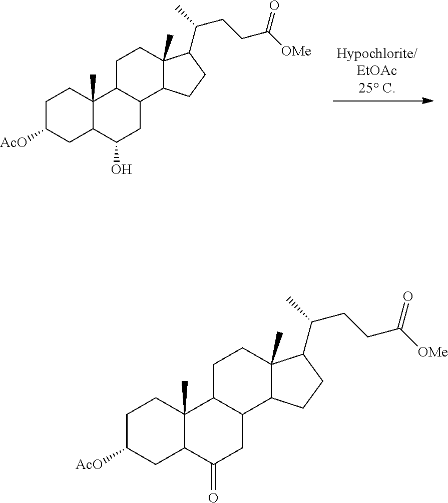 [SMILES: COC(=O)CC[C@@H](C)C1CCC2C3C[C@H](O)C4C[C@H](OC(C)=O)CC[C@]4(C)C3CC[C@@]21C ; COC(=O)CC[C@@H](C)C1CCC2C3CC(=O)C4C[C@H](OC(C)=O)CC[C@]4(C)C3CC[C@@]21C]

0.470 kg of 3′Ac-HDCA-Me from example 8 were dissolved in 2.65 litres of ethyl acetate (technical grade) and 0.66 litres of glacial acetic acid. 2.65 litres of sodium hypochlorite solution (5-10% technical grade) were added to the reaction with cooling so that the reaction temperature did not exceed 20° C. Upon complete conversion to the oxo compound the aqueous phase was discharged and the organic phase washed with 0.8 litres of a 10% sodium dithionite solution. The organic phase was washed with 3.5 litres of water and subsequently dried over magnesium sulfate. The dried organic phase was concentrated to dryness under vacuum to obtain 3′-acetyl-7-oxo-hyodeoxycholic acid methyl ester (3′Ac-7-oxo-HDCA-Me) as a solid.

Yield: 0.391 kg of 3′Ac-7-oxo-HDCA-Me; 83% based on employed 3′Ac-HDCA-Me.

### Example 10—Production of Lithocholic Acid

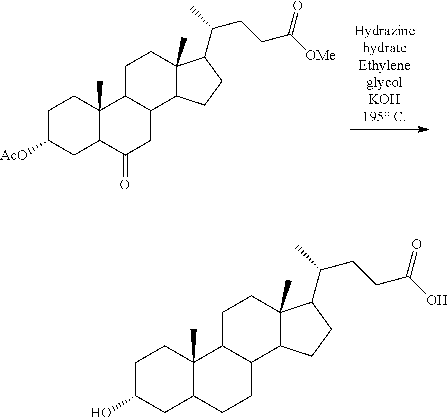 [SMILES: COC(=O)CC[C@@H](C)C1CCC2C3CC(=O)C4C[C@H](OC(C)=O)CC[C@]4(C)C3CC[C@@]21C ; C[C@H](CCC(=O)O)C1CCC2C3CCC4C[C@H](O)CC[C@]4(C)C3CC[C@@]21C]

0.391 kg of 3′-acetyl-7-oxo-hyodeoxycholic acid methyl ester (3′Ac-7-oxo-HDCA-Me) from example 9 were suspended in 1.5 litres of ethylene glycol and 0.425 litres of water were added with stirring. 0.489 kg of solid potassium hydroxide and 4.1 litres of hydrazine hydrate were added to the reaction solution (50% in water). The reaction solution was heated to 130° C. and water and hydrazine hydrate were removed by distillation. Once distillative removal was complete the temperature was set to 195° C. and maintained for 2.5 h. A strong evolution of gas, indicating the progress of the reaction, was observed. The reaction solution was subsequently cooled to below 100° C. and 8.5 litres of a water/ice mixture was then added to the reaction and stirred vigorously. The mixture was then acidified to pH 1 with 0.638 litres of concentrated sulfuric acid. The crude product precipitated as a fine white solid and was filtered off. The crude product was washed with 0.5 litres of water and 0.5 litres of acetonitrile and then dried. The crude lithocholic acid was dissolved in 1.0 L of glacial acetic acid and slowly crystallized by addition of 1.0 litres of water. The produced lithocholic acid was filtered and dried.

Yield: 0.234 kg of lithocholic acid; 60% based on employed 3′Ac-7-oxo-HDCA-Me.

### Example 11—Production of 3′-Acetyl-7-Propylthio-Hyodeoxycholic Acid Methyl Ester

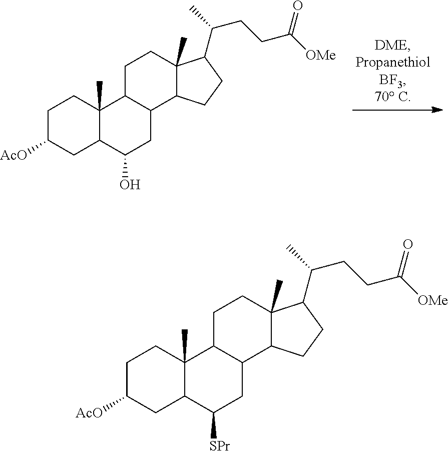

0.470 kg of 3′Ac-HDCA-Me from example 8 were dissolved in 4.5 litres of ethylene glycol dimethyl ether (DME) and 0.337 litres of propanethiol and 0.135 litres of BF₃×Et₂O were added. The reaction solution was heated under reflux for 2 days. The cooled reaction solution was then washed to neutrality with sodium carbonate solution and the organic phase was concentrated to dryness under vacuum to obtain 3′-acetyl-7-propylthio-hyodeoxycholic acid methyl ester as a solid.

Yield: 0.428 kg of 3′-acetyl-7-propylthio-hyodeoxycholic acid methyl ester; 91% based on employed 3′Ac-HDCA-Me.

### Example 12—Production of Lithocholic Acid

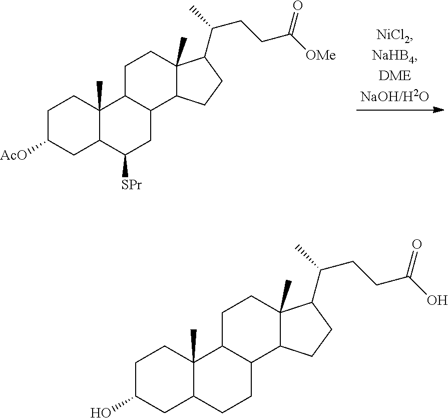

0.428 kg of 3′-acetyl-7-propylthio-hyodeoxycholic acid methyl ester from example 11 and 1.3 kg of nickel chloride hexahydrate were dissolved in 10 litres of methanol-THF (1:1) at 0° C. 0.318 kg of sodium borohydride were added to the reaction solution in small portions of 20 g. Once addition was complete the solution was stirred for a further 30 min. The precipitate was filtered over celite and washed further with methanol-THF. The solvent was removed to dryness under vacuum to obtain crude lithocholic acid as a solid.

The crude lithocholic acid was dissolved in 1.0 L of glacial acetic acid and slowly crystallized by addition of 1.0 litres of water. The produced lithocholic acid was filtered and dried.

Yield: 0.291 kg of lithocholic acid; 68% based on employed 3′-acetyl-7-propylthio-hyodeoxycholic acid methyl ester.

1. A process for producing bile acid derivatives having a protected hydroxyl group in the 3 position comprising:
i) providing a first composition comprising at least one bile acid derivative of general formula I: 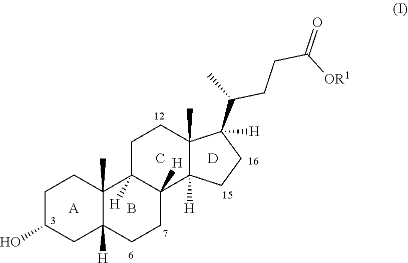
wherein the radical R¹ is selected from the group consisting of C1- to C30-alkyl group, C1- to C30-alkenyl group, C1- to C30-alkynyl group, C5- to C12-cycloalkyl group and C5- to C12-aryl group, wherein the ring B of the bile acid derivative of general formula I has one or two further hydroxyl group(s) at position 6 or at positions 6 and 7 respectively; and wherein none of the rings A, C and D has further hydroxyl groups;
ii) contacting the first composition comprising at least one bile acid derivative of general formula I from i) with a compound R²—X, wherein R² is a —C(═O)—C1- to C30-alkyl group and X is selected from the group consisting of hydroxyl group, —O—C1- to C20-alkyl group, —O—C1- to C20-alkenyl group, —O—C1- to C20-alkynyl group; thiol group, —S—C1- to C20-alkyl group, amine group, —NHR³ group, —NR³R⁴ group, wherein R³ and R⁴ are each independently a C1- to C20-alkyl group, halogen atom and —O—(C═O)—R⁵ group, wherein R⁵ is a C1- to C30-alkyl group; and a lipase selected from the group consisting of lipase B from Candida antarctica of SEQ ID no. 1, lipase 1 from Diutina rugosa of SEQ ID no. 2, lipase 2 from Diutina rugosa of SEQ ID no. 3, lipase 3 from Diutina rugosa of SEQ ID no. 4, lipase 4 from Diutina rugosa of SEQ ID no. 5, lipase 5 from Diutina rugosa of SEQ ID no. 6, lipase from Rhizopus niveus of SEQ ID no. 7, lipase from Aspergillus niger of SEQ ID no. 8 and lipase from Penicillium camemberti FM 013 of SEQ ID no. 9 or a homologous enzyme having a sequence identity of at least 65% with one of the sequences of SEQ ID no. 1 to SEQ ID no. 9 and having the same function as the lipase of SEQ ID no. 1 to SEQ ID no. 9, to obtain a second composition comprising at least one bile acid derivative of general formula II: 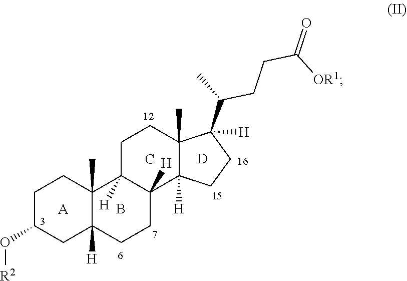
wherein the radical R¹ is as defined at i) for formula I and the radical R² is as defined at ii), wherein the ring B of the bile acid derivative of general formula II has one or two further hydroxyl group(s) at position 6 or at positions 6 and 7 respectively; and wherein none of the rings A, C and D has further hydroxyl groups.

2. The process as claimed in claim 1, wherein the radical R¹ is selected from the group consisting of C1- to C18-alkyl group, C5- to C7-cycloalkyl group and phenyl group, more preferably of C1- to C5-alkyl group, more preferably is an unbranched C1- to C3-alkyl radical, more preferably a methyl radical.

3. The process as claimed in claim 1, wherein X is selected from the group consisting of hydroxyl group, —O—C1- to C20-alkyl group, —O—C1- to C20-alkenyl group, —O—C1- to C20-alkynyl group, thiol group, —S—C1- to C20-alkyl group, amine group, —NHR³ group and —NR³R⁴ group, wherein R³ and R⁴ are each independently a C1- to C20-alkyl group, preferably from the group consisting of hydroxyl group, —O—C1- to C20-alkyl group and —O—C1- to C20-alkenyl group.

4. The process as claimed in claim 1, wherein the radical R² is an unbranched —C(═O)—C1- to C18-alkyl group, preferably an unbranched —C(═O)—C1- to C5-alkyl group, more preferably —C(═O)—CH₃.

5. The process as claimed in claim 1, wherein the lipase employed in ii) is lipase B from Candida antarctica of SEQ ID no. 1 or a homologous enzyme having a sequence identity of at least 65% with the sequence of SEQ ID no. 1 and having the same function as the lipase B from Candida antarctica of SEQ ID no. 1.

6. The process as claimed in claim 1, wherein the homologous enzyme has a sequence identity of at least 80%, preferably of at least 90%, more preferably of at least 95%, more preferably of at least 98%, with the sequence of SEQ ID no. 1 to SEQ ID no. 9 and the same function as the lipase.

7. The process as claimed in claim 1, wherein the ring B of the bile acid derivative of general formula I and of the bile acid derivative of general formula II has one or two further alpha-hydroxyl group(s) at position 6 or at positions 6 and 7 respectively.

8. The process as claimed in claim 1, wherein the bile acid derivative of general formula I is selected from the group consisting of R¹ esters of hyodeoxycholic acid (HDCA), hyocholic acid (HCA) and mixtures of R¹ esters of hyodeoxycholic acid (HDCA) and hyocholic acid (HCA), wherein R¹ is as defined in claim 1 or 2.

9. A bile acid derivative of general formula II obtained or obtainable by a process as claimed in claim 1.

10. A bile acid derivative of general formula II, 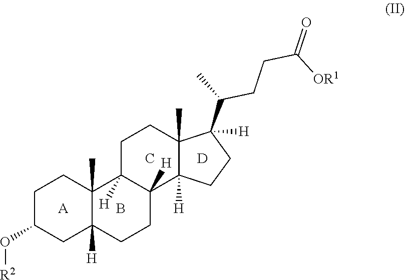
wherein the radical R¹ is selected from the group consisting of C1- to C30-alkyl group, C1- to C30-alkenyl group, C1- to C30-alkynyl group, C5- to C12-cycloalkyl group and C5- to C12-aryl group; and the radical R² is a —C(═O)—C1- to C30-alkyl group, wherein the ring B of the bile acid derivative of general formula II has one or two further hydroxyl group(s) at position 6 or at positions 6 and 7 respectively; and wherein none of the rings A, C and D has further hydroxyl groups.

11. The bile acid derivative as claimed in claim 10 having the formula IIb oder IIc: 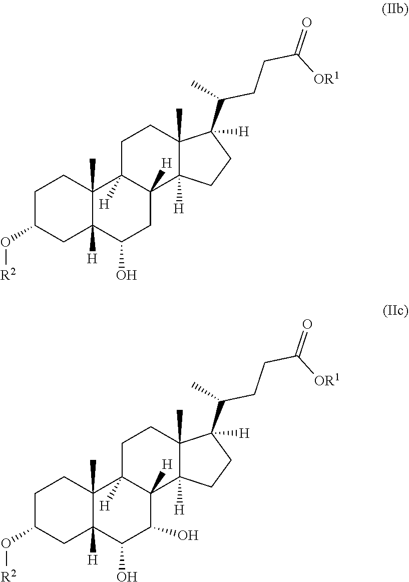
wherein the radical R¹ is in each case selected from the group consisting of C1- to C30-alkyl group, C1- to C30-alkenyl group, C1- to C30-alkynyl group, C5- to C12-cycloalkyl group and C5- to C12-aryl group, preferably from the group consisting of C1- to C18-alkyl group, C5- to C7-cycloalkyl group and phenyl group, more preferably of C1- to C5-alkyl group, more preferably is an unbranched C1- to C3-alkyl radical, more preferably a methyl radical; and the radical R² is a —C(═O)—C1- to C30-alkyl group, preferably an unbranched —C(═O)—C1- to C18-alkyl group, more preferably an unbranched —C(═O)—C1- to C5-alkyl group, more preferably —C(═O)—CH3.

12. The use of a bile acid derivative of general formula II, preferably a bile acid derivative of general formula II obtained or obtainable by the process as claimed in claim 1, 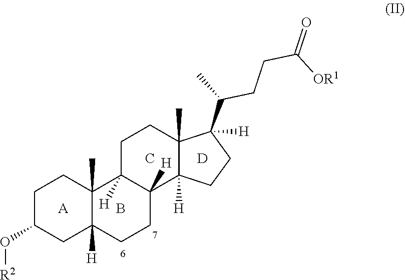
wherein the radical R¹ and the radical R² are as defined in claim 1; wherein the ring B of the bile acid derivative of general formula II has one or two further hydroxyl group(s) at position 6 or at positions 6 and 7 respectively; and wherein none of the rings A, C and D has further hydroxyl groups, for producing lithocholic acid.

13. A process for producing lithocholic acid comprising
i) providing a first composition comprising at least one bile acid derivative of general formula I: 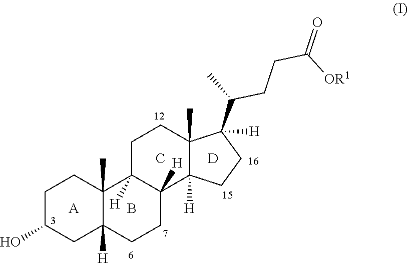
wherein the radical R¹ is selected from the group consisting of C1- to C30-alkyl group, C1- to C30-alkynyl group, C1- to C30-alkenyl group, C5- to C12-cycloalkyl group and C5- to C12-aryl group, wherein the ring B of the bile acid derivative of general formula I has one or two further hydroxyl group(s) at position 6 or at positions 6 and 7 respectively; and wherein none of the rings A, C and D has further hydroxyl groups;
ii) contacting the first composition comprising at least one bile acid derivative of general formula I from i) with a compound R²—X, wherein R² is a —C(═O)—C1- to C30-alkyl group and X is selected from the group consisting of hydroxyl group, —O—C1- to C20-alkyl group, —O—C1- to C20-alkenyl group, —O—C1- to C20-alkynyl group, thiol group, —S—C1- to C20-alkyl group, amine group, —NHR³ group, —NR³R⁴ group, wherein R³ and R⁴ are each independently a C1- to C20-alkyl group, halogen atom and —O—(C═O)—R⁵ group, wherein R⁵ is a C1- to C20-alkyl group; and a lipase selected from the group consisting of SEQ ID no. 1 to SEQ ID no. 9 or a homologous enzyme having a sequence identity of at least 65% with the sequence of SEQ ID no. 1 to SEQ ID no. 9 and having the same function as the lipase of SEQ ID no. 1 to SEQ ID no. 9 to obtain a second composition comprising at least one bile acid derivative of general formula II: 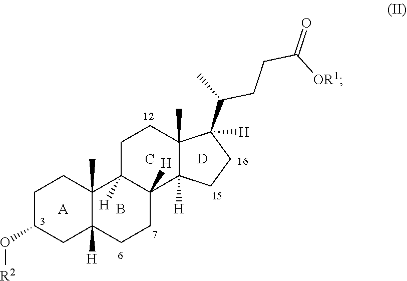
wherein the radical R¹ is as defined at i) for formula I and the radical R² is as defined at ii), wherein the ring B of the bile acid derivative of general formula II has one or two further hydroxyl group(s) at position 6 or at positions 6 and 7 respectively; and wherein none of the rings A, C and D has further hydroxyl groups;
iii) conversion of the bile acid derivative of general formula II obtained from ii) into lithocholic acid.

14. A process for producing lithocholic acid comprising
a) providing a composition comprising a bile acid derivative of general formula IIb, preferably obtained or obtainable by the process as claimed in claim 1, 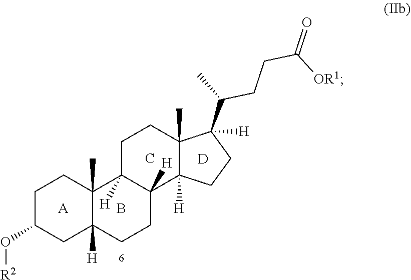
wherein the radical R¹ is selected from the group consisting of C1- to C30-alkyl group, C1- to C30-alkenyl group, C1- to C30-alkynyl group, C5- to C12-cycloalkyl group and C5- to C12-aryl group; the radical R² is a —C(═O)—C1- to C30-alkyl group, wherein the ring B of the bile acid derivative of general formula I has a further hydroxyl group at position 6; and wherein none of the rings A, C and D has further hydroxyl groups;
b) contacting the composition comprising a bile acid derivative of general formula IIb from a) with an oxidant or a C1- to C10-alkylthiol, preferably propanethiol, to convert the at least one hydroxyl group in B and/or D into an ═O group or an —S—C1- to C10-alkyl group, preferably an ═O group or an —S-propyl group to obtain a bile acid derivative of general formula IIIb, 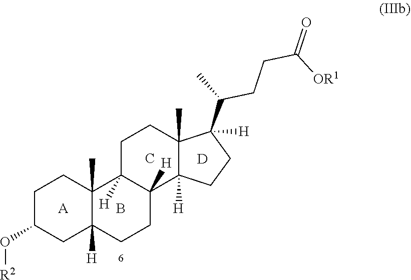
wherein the radical R¹ and the radical R² are as defined in general formula II and the ring B has at least one ═O group or an —S—C1- to C10-alkyl group, preferably an ═O group or an —S-propyl group, at position 6;
c) contacting the bile acid derivative of general formula IIIb from b) with a reducing agent, optionally with additional saponification, to obtain lithocholic acid.

15. A lithocholic acid obtained or obtainable by the process as claimed in claim 13.

16. The use of the lithocholic acid obtained or obtainable by the process as claimed in claim 13 for producing hydroxylated bile acids, preferably ursodeoxycholic acid or ursodeoxycholic acid derivatives.
